(19) United States
(12) Patent Application Publication
(10) Pub. No.: US 2025/0276047 A1
(43) Pub. Date: Sep. 4, 2025

(54) METHODS OF TREATING DISEASES ASSOCIATED WITH TRAUMATIC BRAIN INJURY

(71) Applicant: UNIVERSITY OF SOUTH FLORIDA, Tampa, FL (US)

(72) Inventors: Linda Ines ZOUNGRANA, Tampa, FL (US); Lianchun WANG, Tampa, FL (US)

(21) Appl. No.: 19/067,180

(22) Filed: Feb. 28, 2025

Related U.S. Application Data
(60) Provisional application No. 63/559,989, filed on Mar. 1, 2024.
(60) Provisional application No. 63/717,028, filed on Nov. 6, 2024.

(51) Int. Cl. A61K 38/45; A61K 48/00

(52) U.S. Cl. CPC A61K 38/45; A61K 48/005; C12Y 204/01223

(57) ABSTRACT

Described herein are for treating or preventing conditions, diseases, or disorders related to, associated with, or caused by traumatic brain injury in a subject; treating or preventing a neurodegenerative disease in a subject; and/or reducing Beta-amyloid deposition, tau deposition, or any combination thereof in the brain of a subject. The methods can include administering a therapeutically effective amount of a heparan sulfate proteoglycan modifier agent to a subject.

### CROSS-REFERENCE TO RELATED APPLICATION

This application claims priority to, and the benefit of U.S. Provisional Application 63/559,989, filed on Mar. 1, 2024 and U.S. Provisional Application No. 63/717,028, filed on Nov. 6, 2024, the contents of which are hereby incorporated by reference in their entirety.

### REFERENCE TO SEQUENCE LISTING

The Sequence Listing submitted Feb. 28, 2025, as a text filed named “11001_213US1_SEQUENCE_LISTING” created Feb. 27, 2025, and having a file size of 9.843 bytes is hereby incorporated by reference pursuant to 37 C.F.R. § 1.52 (e)(5).

### BACKGROUND

TBI is one of the leading causes of death and disability worldwide. It has a long-term effect associated with several types of dementia in later life such as AD. TBI mechanism involves the disruption of the blood-brain barrier (BBB) and the secondary injury observed in TBI involves inflammation, oxidative stress, and even an increase in amyloid beta (Aβ), hyperphosphorylated tau (P-Tau), and TDP-43 deposits. Heparan sulfate (HS) is a highly sulfated linear polysaccharide and plays crucial roles in modulating cell-cell and cell-matrix interactions and signaling through its interactions with various protein ligands. It is present on the cell surface and has a protective effect.

There is a need for methods to treat or prevent conditions, diseases, or disorders related to, associated with, or caused by traumatic brain injury. The compositions and methods disclosed herein address these and other needs.

### SUMMARY

Described herein are methods for treating or preventing conditions, diseases, or disorders related to, associated with, or caused by traumatic brain injury in a subject.

Described herein are methods of reducing Beta-amyloid deposition, tau deposition, or any combination thereof in the brain of a subject.

Described herein are also methods of treating or preventing a neurodegenerative disease in a subject.

In some embodiments, the methods can include administering a therapeutically effective amount of a heparan sulfate proteoglycan modifier agent to a subject.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIGS. 1A-1G show recapitulating reduced bEC-Ext1 expression exacerbates Aβ deposition in 5xFAD AD mouse model and decreased bECs to uptake Aβ fibrils. (1A) Analysis of scRNA-seq data (GEO/synapse number: syn18485175; n=24 AD or control per group) shows a 52% reduction in Ext1 expression in bECs from individuals with late-stage AD (p=0.038), with no significant changes in other brain cell types. (1B) Co-immunostaining reveals abundant bEC-HS expression in wild-type (WT) mice (white arrowheads), which was markedly reduced in ecExt1^{+/-} bECs. (1C-1E). 5xFAD; ecExt1^{+/-} mice were induced with tamoxifen (TAM) at 3.5 months and analyzed at 6.5 months using curcumin staining (for aggregated Aβ) and αSMA co-staining. Aβ deposition in arterial walls and parenchyma is indicated by white and red arrows, respectively (1C). Data are shown as means±SEM (1D, 1E). N=3-7 mice per group. (1F, 1G) Aβ uptake by bECs in capillaries was assessed in mouse brain slices (200 μm) incubated with HiLyte™ Fluor 488-Aβ40 (5 μg/ml) for 60 min, with or without heparin (100 μg/ml), followed by CD31 staining. LRP1 is the major Aβ receptor in bECs and endothelial-specific LRP1 knockout mice (Tie2Cre⁺; LRP1^{-/-}) were an additional control. Data are presented mean±SEM. N=3-7 mice per group.

FIGS. 2A-2F. show the rTBI mouse model shows motor function deficit, vascular damage, and reduced HS expression. (2A) Schematic of the optimized rTBI protocol. (2B) Rotarod test on Days 1 (D1), 3 (D3), and 21 (D21) post-rTBI was used to evaluate motor function. (2C-2F) Brain sections from D3 post-rTBI were stained for bECs with tomato lectin (LECTIN) and for HS with anti-HS antibody 10E4, followed by quantification. rTBI brains showed reduced EC staining (2D), lower HS staining (2E), and decreased co-localization of ECs and HS (2F), indicating reduced EC-HS expression. Each dot represents an individual mouse. Data are shown as mean±SEM. Statistical comparisons were made using unpaired Student's t-tests, with P<0.05 considered significant. Scale bar: 1 mm.

FIGS. 3A-3G show reducing bEC-HS exacerbates TBI and AD pathology.
(3A) Rotarod test results for Day 3 (D3) post-TBI mice. (3B) Hanging wire test results for mice at D3 post-TBI. (3C-3E) Immunostaining for ECs (CD31) and vascular tight junction proteins zonula occludens-1 (ZO1) and claudin-5 (CLDN5) with quantification (3D, 3E). (3F, 3G) Immunostaining for mouse Aβ and Tau (Tau-5) with quantification. Each dot represents one mouse. Data are presented as mean±SEM. Statistical comparisons were conducted using unpaired Student's t-tests, with significance set at P<0.05. Scale bar: 50 μm.

FIGS. 4A-4C show results for (4A) AAV2-BR1-GFP expression in bECs in mouse brain. AAV2-Cre-BR1-GFP particles were injected intracranially into postnatal day 3 mice. Four weeks later, brains were stained for bECs using CD31. (4B-4C) AAV2-BR1-iCre-Ext1 vector (Ext1 OE) with AAV2-BR1-iCre-GFP vector as control (Ctr) was injected retro-orbitally into 5xFAD mice at 1.5 month of age and examined at 6 months. Insoluble Aβ40 (4B) and Aβ42 (4C) in whole brain homogenate was quantified using Luminex assay. N=6-11 per group. Data are shown as mean±SD.

FIGS. 5A-5C show inflammation after TBI. Immunostaining images (5A) and graphs (5B-5C) showing the amount of IBA (5B) and FGAP (5C) in cortex and hippocampus for sham and days 1, 3, 8, and 21. The amount of IBA microglial markers increases after day 8 in the cortex of the mice and GFAP increases after day 21. While IBA increases in the hippocampus by days 1, and 3 but starts decreasing while GFAP stays constant.

FIGS. 6A-6C show AD Pathology after TBI. Immunostaining images (6A) and graphs (6B-6C) showing the amount of Aβ (6B) and Tau (6C) in the cortex and hippocampus. The amount of Aβ is higher in the cortex compared to the hippocampus, while Tau remains the same in the cortex and hippocampus.

FIG. 7 show immunostaining images for the Change in the capillaries on the side of injury compared to the side of no injury.

FIGS. 8A-8E show alter protein expression after rTBI in mice at day 3 post-operation (8A) Immunostaining of the vasculature membrane (LECTIN), heparan sulfate (HS), and low-density lipoprotein receptor-related protein 1 (LRP1). Staining quantification of (8B) HS, (8C) LRP1, (8D) Lectin and (8E) EC-HS Mean±SEM, *, P<0.05, ***, P<0.001; NS, non-significant (P>0.05) n=3, student t-test unpair test was conducted.

FIGS. 9A-9F show control wild-type mice motor behavior evaluation comparing sham and TBI group. (9A) and (9B) Rotarod training test, (9C) Rotarod test, (9D) Hanging wire test were performed on days 1, 8, and 21 post-operation in rTBI on WT mice and (9E) and (9F) Fear Conditioning. Mean±SEM, *, P<0.05, **, P<0.01; ***, P<0.001; NS, non-significant (P>0.05) n=2, 3 and 4, two-way ANOVA test was conducted.

FIGS. 10A-10F show vascular dysfunction after rTBI in both wild-type and EXT1 Knockout mice comparing sham and day 3 post-operation (10A) Immunostaining of vasculature endothelium cell (CD31), and astrocytes (AQP4) making up the vasculature, (10B) Average vasculature branching, (10C) average number of branches (10D) vasculature density, (10E) vasculature total area, (10F) relative staining intensity of AQP4 over CD31. Mean±SEM, *, P<0.05, **, P<0.01; ***, P<0.001; NS, non-significant (P>0.05) n=4, two-way ANOVA test was conducted.

Like reference symbols in the various drawings indicate like elements.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

### General Definitions

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. A range may be construed to include the start and the end of the range. For example, a range of 10% to 20% (i.e., range of 10%-20%) can includes 10% and also includes 20%, and includes percentages in between 10% and 20%, unless explicitly stated otherwise herein.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inactivate”, “inactivating” and “inactivation” means to decrease or eliminate an activity, response, condition, disease, or other biological parameter due to a chemical (covalent bond formation) between the ligand and a its biological target.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage. In particular, the term “treatment” includes the alleviation, in part or in whole, of the symptoms of coronavirus infection (e.g., sore throat, blocked and/or runny nose, cough and/or elevated temperature associated with a common cold). Such treatment may include eradication, or slowing of population growth, of a microbial agent associated with inflammation.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event. In particular embodiments, “prevention” includes reduction in risk of coronavirus infection in patients. However, it will be appreciated that such prevention may not be absolute, i.e., it may not prevent all such patients developing a coronavirus infection, or may only partially prevent an infection in a single individual. As such, the terms “prevention” and “prophylaxis” may be used interchangeably.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective’ amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

### Biological Definitions

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers. In some embodiments, the polynucleotide is composed of nucleotide monomers of generally greater than 100 nucleotides in length and up to about 8,000 or more nucleotides in length.

Nucleic acids may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or chimeras or combinations thereof.

In some embodiments, polynucleotides of the present disclosure function as messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. The skilled artisan will appreciate that, except where otherwise noted, polynucleotide sequences set forth in the instant application will recite “T”s in a representative DNA sequence but where the sequence represents RNA (e.g., mRNA), the “T”s would be substituted for “U”s. Thus, any of the RNA polynucleotides encoded by a DNA identified by a particular sequence identification number may also comprise the corresponding RNA (e.g., mRNA) sequence encoded by the DNA, where each “T” of the DNA sequence is substituted with “U.”

The basic components of an mRNA molecule typically include at least one coding region, a 5′ untranslated region (UTR), a 3′ UTR, a 5′ cap and a poly-A tail. Polynucleotides of the present disclosure may function as mRNA but can be distinguished from wild-type mRNA in their functional and/or structural design features, which serve to overcome existing problems of effective polypeptide expression using nucleic-acid based therapeutics.

In some embodiments, a RNA polynucleotide of an RNA (e.g., mRNA) vaccine encodes 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9 or 9-10 antigenic polypeptides. In some embodiments, a nucleic acid sequence (e.g., mRNA) encodes at least 10, 20, 30, 40, 50, 60, 70, 80, 90 or 100 antigenic polypeptides. In some embodiments, a nucleic acid sequence (e.g., mRNA) encodes at least 100 or at least 200 antigenic polypeptides. In some embodiments, a RNA polynucleotide of an encodes 1-10, 5-15, 10-20, 15-25, 20-30, 25-35, 30-40, 35-45, 40-50, 1-50, 1-100, 2-50 or 2-100 antigenic polypeptides.

Polynucleotides of the present disclosure, in some embodiments, are codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art—non-limiting examples include services from GeneArt (Life Technologies), DNA2.0 (Menlo Park Calif.) and/or proprietary methods. In some embodiments, the open reading frame (ORF) sequence is optimized using optimization algorithms.

In some embodiments, a codon optimized sequence shares less than 95% sequence identity, less than 90% sequence identity, less than 85% sequence identity, less than 80% sequence identity, or less than 75% sequence identity to a naturally-occurring or wild-type sequence (e.g., a naturally-occurring or wild-type mRNA sequence encoding a polypeptide or protein of interest (e.g., an antigenic protein or antigenic polypeptide)).

In some embodiments, a codon-optimized sequence shares between 65% and 85% (e.g., between about 67% and about 85%, or between about 67% and about 80%) sequence identity to a naturally-occurring sequence or a wild-type sequence (e.g., a naturally-occurring or wild-type mRNA sequence encoding a polypeptide or protein of interest (e.g., an antigenic protein or polypeptide)). In some embodiments, a codon-optimized sequence shares between 65% and 75%, or about 80% sequence identity to a naturally-occurring sequence or wild-type sequence (e.g., a naturally-occurring or wild-type mRNA sequence encoding a polypeptide or protein of interest (e.g., an antigenic protein or polypeptide)).

In some embodiments a codon-optimized RNA (e.g., mRNA) may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

The term “polypeptide” refers to a compound made up of a single chain of D- or L-amino acids or a mixture of D- and L-amino acids joined by peptide bonds.

In some embodiments, a polypeptide is longer than 25 amino acids and shorter than 50 amino acids. The term “antigenic polypeptide” includes full length polypeptides/proteins as well as immunogenic fragments thereof (immunogenic fragments capable of inducing an immune response to an infection agent). Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer, or tetramer. Polypeptides may also comprise single chain or multichain polypeptides such as antibodies or insulin and may be associated or linked. Most commonly, disulfide linkages are found in multichain polypeptides. The term polypeptide may also apply to amino acid polymers in which at least one amino acid residue is an artificial chemical analogue of a corresponding naturally-occurring amino acid.

A “polypeptide variant” is a molecule that differs in its amino acid sequence relative to a native sequence or a reference sequence. Amino acid sequence variants may possess substitutions, deletions, insertions, or a combination of any two or three of the foregoing, at certain positions within the amino acid sequence, as compared to a native sequence or a reference sequence. Ordinarily, variants possess at least 50% identity to a native sequence or a reference sequence. In some embodiments, variants share at least 80% identity or at least 90% identity with a native sequence or a reference sequence.

In some embodiments “variant mimics” are provided. A “variant mimic” contains at least one amino acid that would mimic an activated sequence. For example, glutamate may serve as a mimic for phosphoro-threonine and/or phosphoro-serine. Alternatively, variant mimics may result in deactivation or in an inactivated product containing the mimic. For example, phenylalanine may act as an inactivating substitution for tyrosine, or alanine may act as an inactivating substitution for serine.

“Orthologs” refers to genes in different species that evolved from a common ancestral gene by speciation. Normally, orthologs retain the same function in the course of evolution. Identification of orthologs is important for reliable prediction of gene function in newly sequenced genomes.

“Analogs” is meant to include polypeptide variants that differ by one or more amino acid alterations, for example, substitutions, additions or deletions of amino acid residues that still maintain one or more of the properties of the parent or starting polypeptide.

The present disclosure provides several types of compositions that are polynucleotide or polypeptide based, including variants and derivatives. These include, for example, substitutional, insertional, deletion and covalent variants and derivatives. The term “derivative” is synonymous with the term “variant” and generally refers to a molecule that has been modified and/or changed in any way relative to a reference molecule or a starting molecule.

As such, polynucleotides encoding peptides or polypeptides containing substitutions, insertions and/or additions, deletions and covalent modifications with respect to reference sequences, in particular the polypeptide sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal residues or N-terminal residues) alternatively may be deleted depending on the use of the sequence, as for example, expression of the sequence as part of a larger sequence that is soluble, or linked to a solid support.

“Substitutional variants” when referring to polypeptides are those that have at least one amino acid residue in a native or starting sequence removed and a different amino acid inserted in its place at the same position. Substitutions may be single, where only one amino acid in the molecule has been substituted, or they may be multiple, where two or more (e.g., 3, 4 or 5) amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Features” when referring to polypeptide or polynucleotide are defined as distinct amino acid sequence-based or nucleotide-based components of a molecule respectively. Features of the polypeptides encoded by the polynucleotides include surface manifestations, local conformational shape, folds, loops, half-loops, domains, half-domains, sites, termini and any combination(s) thereof.

As used herein when referring to polypeptides the term “domain” refers to a motif of a polypeptide having one or more identifiable structural or functional characteristics or properties (e.g., binding capacity, serving as a site for protein-protein interactions).

As used herein when referring to polypeptides the terms “site” as it pertains to amino acid based embodiments is used synonymously with “amino acid residue” and “amino acid side chain.” As used herein when referring to polynucleotides the terms “site” as it pertains to nucleotide based embodiments is used synonymously with “nucleotide.” A site represents a position within a peptide or polypeptide or polynucleotide that may be modified, manipulated, altered, derivatized or varied within the polypeptide-based or polynucleotide-based molecules.

As recognized by those skilled in the art, protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein having a length of 10, 20, 30, 40, 50, 60, 70, 80, 90, 100 or longer than 100 amino acids. In another example, any protein that includes a stretch of 20, 30, 40, 50, or 100 (contiguous) amino acids that are 40%, 50%, 60%, 70%, 80%, 90%, 95%, or 100% identical to any of the sequences described herein can be utilized in accordance with the disclosure. In some embodiments, a polypeptide includes 2, 3, 4, 5, 6, 7, 8, 9, 10, or more mutations as shown in any of the sequences provided herein or referenced herein. In another example, any protein that includes a stretch of 20, 30, 40, 50, or 100 amino acids that are greater than 80%, 90%, 95%, or 100% identical to any of the sequences described herein, wherein the protein has a stretch of 5, 10, 15, 20, 25, or 30 amino acids that are less than 80%, 75%, 70%, 65% to 60% identical to any of the sequences described herein can be utilized in accordance with the disclosure.

Polypeptide or polynucleotide molecules of the present disclosure may share a certain degree of sequence similarity or identity with the reference molecules (e.g., reference polypeptides or reference polynucleotides), for example, with art-described molecules (e.g., engineered or designed molecules or wild-type molecules). The term “identity,” as known in the art, refers to a relationship between the sequences of two or more polypeptides or polynucleotides, as determined by comparing the sequences. In the art, identity also means the degree of sequence relatedness between two sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. Identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al. (1997). “Gapped BLAST and PSI-BLAST: a new generation of protein database search programs,” Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently, a Fast Optimal Global Sequence Alignment Algorithm (FOGSAA) was developed that purportedly produces global alignment of nucleotide and protein sequences faster than other optimal global alignment methods, including the Needleman-Wunsch algorithm. Other tools are described herein, specifically in the definition of “identity” below.

As used herein, the term “homology” refers to the overall relatedness between polymeric molecules, e.g. between nucleic acid molecules (e.g. DNA molecules and/or RNA molecules) and/or between polypeptide molecules. Polymeric molecules (e.g. nucleic acid molecules (e.g. DNA molecules and/or RNA molecules) and/or polypeptide molecules) that share a threshold level of similarity or identity determined by alignment of matching residues are termed homologous. Homology is a qualitative term that describes a relationship between molecules and can be based upon the quantitative similarity or identity. Similarity or identity is a quantitative term that defines the degree of sequence match between two compared sequences. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). Two polynucleotide sequences are considered homologous if the polypeptides they encode are at least 50%, 60%, 70%, 80%, 90%, 95%, or even 99% for at least one stretch of at least 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered homologous if the proteins are at least 50%, 60%, 70%, 80%, or 90% identical for at least one stretch of at least 20 amino acids.

Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function in the course of evolution. “Paralogs” are genes (or proteins) related by duplication within a genome. Orthologs retain the same function in the course of evolution, whereas paralogs evolve new functions, even if these are related to the original one.

The term “identity” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

The term “complementary” refers to the topological compatibility or matching together of interacting surfaces of a probe molecule and its target. Thus, the target and its probe can be described as complementary, and furthermore, the contact surface characteristics are complementary to each other.

The term “hybridization” refers to a process of establishing a non-covalent, sequence-specific interaction between two or more complementary strands of nucleic acids into a single hybrid, which in the case of two strands is referred to as a duplex.

The term “anneal” refers to the process by which a single-stranded nucleic acid sequence pairs by hydrogen bonds to a complementary sequence, forming a double-stranded nucleic acid sequence, including the reformation (renaturation) of complementary strands that were separated by heat (thermally denatured).

The term “melting” refers to the denaturation of a double-stranded nucleic acid sequence due to high temperatures, resulting in the separation of the double strand into two single strands by breaking the hydrogen bonds between the strands.

The term “target” refers to a molecule that has an affinity for a given probe. Targets may be naturally-occurring or man-made molecules. Also, they can be employed in their unaltered state or as aggregates with other species.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It is appreciated by those skilled in the art that the design of the expression vector can depend on such factors as the choice of the host cell to be transformed, the level of expression desired, etc.

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

The term “expression cassette” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

A nucleic acid sequence is “heterologous” to a second nucleic acid sequence if it originates from a foreign species, or, if from the same species, is modified by human action from its original form. For example, a heterologous promoter (or heterologous 5′ untranslated region (5′UTR)) operably linked to a coding sequence refers to a coding sequence from a species different from that from which the promoter was derived, or, if from the same species, a coding sequence which is different from naturally occurring allelic variants (for example, the 5′UTR or 3′UTR from a different gene is operably linked to a nucleic acid encoding for a co-stimulatory molecule).

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or antigen binding fragment thereof” or “antibody or fragments thereof” encompasses chimeric antibodies and hybrid antibodies, with dual or multiple antigen or epitope specificities, and fragments, such as F(ab′)2, Fab′, Fab, Fv, sFv, scFv and the like, including hybrid fragments. Thus, fragments of the antibodies that retain the ability to bind their specific antigens are provided. For example, fragments of antibodies which maintain binding activity are included within the meaning of the term “antibody or antigen binding fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or antigen binding fragment thereof” are conjugates of antibody fragments and antigen binding proteins (single chain antibodies). Also included within the meaning of “antibody or antigen binding fragment thereof” are immunoglobulin single variable domains, such as for example a nanobody.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

As used herein, “immune effector cells” refers to cells capable of binding an antigen or a peptide and which mediate an immune response. These cells include, but are not limited to, T cells (include CD4+ and CD8+ T cells), B cells, monocytes, macrophages, NK cells and cytotoxic T lymphocytes (CTLs).

Reference will now be made in detail to specific aspects of the disclosed materials, comp ounds, compositions, articles, and methods, examples of which are illustrated in the accompanying Examples and Figures.

### Methods

Described is a method for treating or preventing conditions, diseases, or disorders related to, associated with, or caused by traumatic brain injury in a subject, the method including: administering a therapeutically effective amount of a heparan sulfate proteoglycan modifier agent to a subject.

In some embodiments, the condition, disease, or disorder related to, associated with, or caused by traumatic brain injury is a neurodegenerative disease.

In some embodiments, the method reduces Beta-amyloid deposition, tau deposition, or any combination thereof of a subject in need thereof.

Described herein are methods of reducing Beta-amyloid deposition, tau deposition, or any combination thereof in the brain of a subject, the method including: administering a therapeutically effective amount of a heparan sulfate proteoglycan modifier agent to a subject.

Described herein are also methods of treating or preventing a neurodegenerative disease in a subject, the method including: administering a therapeutically effective amount of a heparan sulfate proteoglycan modifier agent to a subject.

In some embodiments, the neurodegenerative disease can include, but are not limited to, Alzheimer's disease (AD), frontotemporal dementia (FTD), progressive supranuclear palsy, corticobasal degeneration (CBD), chronic traumatic encephalopathy (CTE), or any combination thereof.

In some embodiments, the methods can increase the expression of heparan sulfate proteoglycan in brain endothelial cells compared to the expression of heparan sulfate proteoglycan without administration of an effective amount of heparan sulfate proteoglycan modifier agent. In some embodiments, the methods can increase the concentration of heparan sulfate proteoglycan in brain endothelial cells compared to the concentration of heparan sulfate proteoglycan without administration of an effective amount of heparan sulfate proteoglycan modifier agent. In some embodiments, the methods can increase the concentration of exostosin like glycosyltransferase 3 (EXTL3) in brain endothelial cells compared to the concentration of exostosin like glycosyltransferase 3 (EXTL3) without administration of an effective amount of heparan sulfate proteoglycan modifier agent.

In some embodiments, the heparan sulfate proteoglycan modifier agent can be a nucleic acid, a protein/peptide, a small molecule, or any combination thereof. In some embodiments, the heparan sulfate proteoglycan modifier agent can be a nucleic acid. In some embodiments, the nucleic acid can include, but is not limited, to double stranded DNA, single-stranded DNA, complexed DNA, encapsulated DNA, naked RNA, encapsulated RNA, messenger RNA (mRNA), tRNA, short interfering RNA (siRNA), double stranded RNA (dsRNA), micro-RNA (miRNA), a long non-coding RNAs (IncRNAs), antisense RNA (asRNA), self-amplify mRNA (saRNA), guide RNA (gRNA), cRNA, or any combination thereof. In some embodiments, the nucleic acid can be a messenger RNA (mRNA).

In some embodiments, the heparan sulfate proteoglycan modifier agent can include a nucleic acid having at least 80% sequence identity to SEQ ID NO: 1. In some embodiments, the heparan sulfate proteoglycan modifier agent can be protein/peptide. In some embodiments, the protein/peptide can have at least 80% sequence identity to SEQ ID NO: 2.

In some embodiments, the heparan sulfate proteoglycan modifier agent can include a nucleic acid having at least 80% sequence identity to SEQ ID NO: 1 encoding for exostosin like glycosyltransferase 3 (EXTL3), a fragment, or variant thereof having at least 80% sequence identity to SEQ ID NO: 2. In some embodiments, the nucleic acid encodes for exostosin like glycosyltransferase 3 (EXTL3), a fragment, or variant thereof having at least 80% sequence identity to SEQ ID NO: 2.

The active agents (e.g., “heparan sulfate proteoglycan modifier agent”) as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. The active agent may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc. Administration of the active components of their compositions can be a single administration, or at continuous and distinct intervals as can be readily determined by a person skilled in the art.

In certain embodiments, it may be desirable to provide continuous delivery of one or more compounds to a patient in need thereof. For intravenous or intraarterial routes, this can be accomplished using drip systems, such as by intravenous administration. For topical applications, repeated application can be done or a patch can be used to provide continuous administration of the compounds over an extended period of time.

The active ingredient may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications. Dosage can vary, and can be administered in one or more dose administrations daily, for one or several days.

In some embodiments, the methods include administering the active agent and a pharmaceutically acceptable carrier. In other examples, administration can include an excipient.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21 st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene(polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The compounds can be incorporated microparticles, nanoparticles, or combinations thereof that provide controlled release of the compounds and/or additional active agents. For example, the compounds can be incorporated into polymeric microparticles, which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives.

Polymers, which are slowly soluble and form a gel in an aqueous environment, such as hydroxypropyl methylcellulose or polyethylene oxide, may also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the compound can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents may be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins, which are water insoluble, such as zein, can also be used as materials for the formation of drug containing microparticles. Additionally, proteins, polysaccharides and combinations thereof, which are water-soluble, can be formulated with drug into microparticles and subsequently cross-linked to form an insoluble network. For example, cyclodextrins can be complexed with individual drug molecules and subsequently cross-linked.

Encapsulation or incorporation of drug into carrier materials to produce drug-containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. These processes are known in the art.

For some carrier materials it may be desirable to use a solvent evaporation technique to produce drug-containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, drug(s) in a particulate form is homogeneously dispersed in a water-insoluble or slowly water soluble material. To minimize the size of the drug particles within the composition, the drug powder itself may be milled to generate fine particles prior to formulation. The process of jet milling, known in the pharmaceutical art, can be used for this purpose. In some embodiments, drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

The compounds described herein can be incorporated into injectable/implantable solid or semi-solid implants, such as polymeric implants. In one embodiment, the compounds are incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication require polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent.

Alternatively, the compounds can be incorporated into a polymer matrix and molded, compressed, or extruded into a device that is a solid at room temperature. For example, the compounds can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, wafers, or extruded into a device, such as rods.

The release of the compounds from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the compounds from the implant are well known in the art.

In some embodiments, the pharmaceutical compositions can be administered locally. In some embodiments, the compounds are incorporated in a delivery system such as gels, nanoparticles, microparticles, or implants such as (e.g., rods, discs, wafers, orthopedic implants) for sustained release. In some embodiments, the compounds can be administered using a local delivery implantable system comprising the compounds incorporated within a gel, nanoparticles, microparticles, or an implant. In some embodiments, the pharmaceutical compositions comprise a delivery system such as gels, nanoparticles, microparticles, or implants such as (e.g., rods, discs, wafers, orthopedic implants) for sustained release of the active agent or a pharmaceutically acceptable salt or derivative thereof.

Sequence Listing

SEQ ID NO: 1 (EXTL3_HUMAN Nucleic Acid Sequence GenBank: AF001690.1)
GCGGCGGCGGCGGGTCCCTGAGCTGGAAGCCGGAGAGCAAGCCCTGGAGGTTCACT
CTTTCAAGAAGTCGTGTGCTGAGGTGTAATGCTACACAAGTCAGAGGAAGGAAGGG
TCCTGAAACACATGGCCTGATTGTTGGCAAAGGCATCATAAGAAGCTGGCATTTATT
TCTGTTCTAACCTATTACTGTATAACTGTGAATAGACACTATGCATATTTGTTGGTCA
GCAAAACCAAGAAACAAGAGCTATGGCATTTGAAAAAGTCTGTCTGATTCCAGGGT
GTTTTTCCTGGGTTTCATCATCAGGTACCTCCTCCCTTTCATCTCAGCAAGAATGTGG
CACCTTTTATCGTTTGATAAAGATTAAGGACATGTTCTTTGGTCAACAGCCAGAACT
TAAAATCTGCTGGAATAGGGTCAGAGACCATTTCAGCTGCAGCTGAGGAAAATGAA
ATGTTCATTTTATTTGGTGCCTTGTCTGGGGAGCACACTAACTCTTCTGGAAACGTGT
CAGTGAAACAGAGATCGTTTTGTGGAATAGCAACCCATGGTTATGGCGAGTGACCC
GACGTGATCTGGGGGGCAGGCTGCAGAGGACTCATGACAGGCTATACCATGCTGCG
GAATGGGGGCGCGGGGAACGGAGGTCAGACCTGCATGCTGCGCTGGTCCAACCGCA
TCCGCCTCACGTGGCTCAGCTTCACGCTCTTTGTCATCCTGGTCTTCTTCCCGCTCAT
CGCCCACTATTACCTCACCACTCTGGATGAGGCTGATGAGGCAGGCAAGCGGATTTT
TGGTCCCCGGGTGGGGAACGAGCTGTGCGAGGTGAAGCACGTGCTGGATCTGTGCC
GCATCCGGGAGTCGGTGAGTGAAGAGCTCCTGCAGCTGGAGGCCAAGCGCCAAGAG
CTGAACAGCGAGATCGCCAAGCTGAATCTGAAGATCGAAGCCTGTAAGAAGAGCAT
TGAGAACGCCAAGCAGGACCTGCTCCAGCTCAAGAATGTCATCAGCCAGACCGAGC
ATTCCTACAAGGAGCTCATGGCCCAGAACCAGCCCAAGCTGTCCCTGCCCATCCGAC
TGCTCCCAGAGAAGGACGATGCCGGCCTCCCTCCCCCGAAGGCCACTCGGGGCTGC
CGGCTACACAACTGCTTTGATTATTCTCGTTGCCCTCTCACCTCTGGCTTCCCGGTCT
ACGTCTATGACAGTGACCAGTTTGTCTTTGGCAGCTACCTGGATCCCTTGGTCAAGC
AGGCTTTTCAGGCGACAGCACGAGCTAACGTTTATGTTACAGAAAATGCAGACATC
GCCTGCCTTTACGTGATACTAGTGGGAGAGATGCAGGAGCCGGTGGTGCTGCGGCC
TGCTGAGCTGGAGAAGCAGTTGTATTCCCTGCCACACTGGCGGACGGATGGACACA
ACCATGTCATCATCAATCTGTCACGTAAGTCAGATACACAGAACCTTCTCTATAACG
TCAGTACTGGCCGTGCCATGGTGGCCCAGTCCACCTTCTACACTGTCCAGTACAGAC
CTGGCTTTGACTTGGTCGTATCACCGCTGGTCCATGCCATGTCTGAGCCCAACTTCAT
GGAAATCCCACCACAGGTGCCGGTGAAGCGGAAATATCTCTTCACCTTCCAGGGCG
AGAAGATTGAGTCTCTGAGGTCTAGCCTTCAGGAGGCCCGCTCCTTCGAAGAGGAA
ATGGAGGGCGACCCTCCCGCCGACTACGATGACCGGATCATTGCCACCCTGAAGGC
GGTGCAGGACAGCAAGCTGGATCAGGTCCTGGTGGAATTCACCTGCAAAAACCAGC
CCAAACCCAGCCTGCCGACTGAGTGGGCACTGTGTGGAGAGCGGGAGGACCGCTTG
GAATTGCTGAAGCTCTCCACCTTCGCCCTCATCATTACCCCCGGGGACCCTCGCTTG
GTTATTTCCTCTGGGTGTGCAACACGGCTCTTCGAAGCCCTGGAAGTCGGTGCCGTC
CCGGTGGTGCTGGGGGAGCAGGTCCAGCTTCCCTACCAGGACATGCTGCAGTGGAA
CGAGGCGGCCCTGGTGGTGCCAAAGCCTCGTGTTACCGAGGTTCATTTCCTGCTCAG
AAGCCTCTCCGATAGTGACCTCCTGGCTATGAGGCGGCAAGGCCGCTTTCTCTGGGA
GACTTACTTCTCCACTGCTGACAGTATTTTTAATACCGTGCTGGCTATGATTAGGACT
CGCATCCAGATCCCAGCCGCTCCCATCCGGGAAGAGGCGGCAGCTGAGATCCCCCA
CCGTTCAGGCAAGGCGGCTGGAACTGACCCCAACATGGCTGACAACGGGGACCTGG
ACCTGGGGCCAGTGGAGACGGAGCCGCCCTACGCCTCACCCAGATACCTCCGCAAT
TTCACTCTGACTGTCACTGACTTTTACCGCAGCTGGAACTGTGCTCCAGGGCCTTTCC
ATCTTTTCCCCCACACTCCCTTTGACCCTGTGTTGCCCTCAGAGGCCAAATTCTTGGG
CTCAGGGACTGGCTTTCGGCCTATTGGTGGTGGAGCTGGGGGTTCTGGCAAGGAATT
TCAGGCAGCGCTTGGAGGCAATGTTCCCCGAGAGCAGTTCACGGTGGTGATGTTGA
CTTATGAGCGGGAGGAAGTGCTTATGAACTCTTTAGAGAGGCTGAATGGCCTCCCTT
ACCTGAACAAGGTCGTGGTGGTGTGGAATTCTCCCAAGCTGCCATCAGAGGACCTTC
TGTGGCCTGACATTGGCGTCCCCATCATGGTGGTCCGTACTGAGAAGAACAGTTTGA
ACAACCGATTCTTACCCTGGAATGAAATTGAGACAGAGGCCATCCTGTCCATTGATG
ACGATGCTCACCTCCGCCATGACGAAATCATGTTTGGGTTCCGGGTGTGGAGAGAA
GCTCGGGACCGCATCGTGGGCTTCCCTGGCCGTTACCACGCATGGGACATCCCCCAT
CAGTCCTGGCTCTACAACTCCAACTACTCCTGTGAGCTGTCCATGGTGCTGACAGGT
GCTGCCTTCTTTCACAAGTATTATGCCTACCTGTATTCTTATGTGATGCCCCAGGCCA
TCCGGGACATGGTGGATGAATACATCAACTGTGAGGACATTGCCATGAACTTCCTTG
TCTCCCACATCACTCGGAAGCCCCCCATCAAGGTGACCTCACGGTGGACATTCCGAT
GCCCAGGATGCCCTCAGGCCCTGTCTCATGATGACTCCCACTTCCACGAGCGGCACA
AGTGCATCAACTTCTTCGTGAAGGTGTACGGCTACATGCCCCTCCTGTACACGCAGT
TCAGGGTGGATTCTGTGCTCTTCAAGACACGCCTGCCCCATGACAAGACCAAGTGCT
TCAAGTTCATCTAGGGGCAGCGCACGGTCTGGGGAAGAGGATGAGCAGAGGGAGG
AAGATGGCTCCCAAGGTTCCTAGGCATTGCAGGACCTTGGGCACATCTGCTGGTGG
GTGGCCCAGAGCCTCTGCTGGAAGGGGCAGCAGGAGGAGTGGAAGGAAACCGCTG
CCTTTATCTTGAAGTCAGCCACACTGGGCCTGGAGCCCTGGGCGGAGTCCCCGGGGT
TCCCCACACAGGGCACTGACTGATAGCTTACACTGAGGACTGTGGCGACTCTGCAG
AGTCACTCACACCGTTCGTACGCCCAGGACAGCTGGTTCGTGGTTTTTACATTCAAT
AACAACTATTATGATTATTTAAAAAGAGAAAGTTTCAGATTTGCCATTCAAGGCTTA
TTTATATATATGTGTGTGTATATAAATACATGCACACACTTGCATACATATATATTTT
TGGCTGGGGGAGTGTGAGTTTTGCCTTTCTAAGGGAGGGACCGCGCAGGCTCCTTTG
TTCTGTATTCTGGCGGAGATGGGTCCTGGCCTTGTGTCACTGGCTTATCCTTAAAGAT
CATCTCCCATCCTCCCCAGCGCCATCTGTGTGCAGCAACCAGAAAGGGATGAACTTG
GCCCTCTTGCGGGCCTGGACAAGGTCTCTTCCTTACCCTTTCTGTTGCCAGTCAGCAA
CCTGTAACTCACATTCTCTTCCCAGTGAATCCCTGGGAGCGCCTGACCCTGGTGGGC
TGTTCAGCTTCCTGCTGCTGGGGCCAGCGATTTTTGAGGATTTATCTTTAGGCCAGG
CTTGCCTCCGTACTTATCCCTGCTCTCCCATTTCTCTCTTGTTTGAGAGAGAATGAGG
AAGCAAAGAGTGAGAAAGAATAGGGGCTGAAGACGCCACTCCCAGATGGCTCTTTC
TATCCTGCTCTTCTGTTGAAACACACGTGCTGTGGGCCTCAGGCGTTTCTGAAGTGC
TCTTTCTTGGATTGGACAGGAGATCAGCAGCGTGCACATCTGCTGTGGTCTGAAGTG
GTTTGCAGGTCAGCCTCCTCTCCCTAGTGTAGAGCAAGCCAGTGTCCTTCGAGGAAC
CCACCCGGCTGGCCGGGAAGTTTTACAGCAAGGCGCCTGCCTTGGGATAATTCCTTG
GTGAAATTCACCTTCCCCCCGCCTCTGTCTGGAGCCCCATCCTGTGTTATCTGTGGTT
TTTGGACCCCTAATGTCAGCTTGGCTGTAGGACTCCCCGAGGTTTGGTATGTGCTAG
AACAATGGGAGGCTGTGATTTGCTGTGTAAGCTCACATCCAGCCTTGGAATCTAACG
GGCATTCACAACCCGAGTTACCACTTTCCACTCCCTGCTTAGGATTCTGTTCCCTGGG
CTGAAACTGAAATAAGCTAATTTTTTGGGTCACGGTGGCAGTAGGGGAACCTAGGA
GGGTGTGAGTGGCATTTGTCAGGGATTTAGCCCATGACGTGTTTCTTGAACCCTACT
TTCTGGAAGTGGAGTTGACTCTGGAAGTTTTCTAGCAACTGAACAAAAGCTCAGGTT
TGTCCTGGTCATGCACATGCCTTAAGCCAGTTCCGTCTTCCCTAGACCTTGGCATCCT
GTGCTTCTATTTCTTGGAATACGTTCTCCTCTGACCTGCCTGTACCACGTGGGTCCTC
TTCAAGTACTGTTTTGAAGCTGGGCTCTTTTGTGTAGCTCCCACCCACCTGTAGGGCT
AGCTCGGCTTAAGGGAACTCTCCCCATTGGCAAACCGGACCCGGCCGCCGCCAGGA
CTGTGTTTCCAAAGGTTCCCCGCCCCCAACCCCAGCATCAGCCTGTAGCTCCCCTGC
TGAGGCAGTGTGGTTATGTTCCCAGCAGTGGGGGTCAGACGCCCTTCCTCAGAACTT
TCTAGTTGCCCTCTACCTGACTCCTGACTTGTATTCCTTTTAGCAGTAGCCTTCTTCC
CTCGGGGAGCCAAAGAGTGTGGTGTGTGGCGCTATATTGTGGCTGCTATTTCATCTG
GTTTCTTTTAATGTGAGGAACTCACATACTGACTTCAGTGGGACTCGGTGAGCCGGG
GCCGTCTGTGTGGTGGGACCCCCTTTAGCGGGACTCAGTGAGCTGGGGCCGTCTGTG
TGGTGGAGCCAGGGCCTCTCCCTTTAGTGGAGCCAGGTTGTCGGGCCCCGAATGTCA
CTGGTGGATCTAAGAAGGGCTGAGTGGTCTGACACCAAAACATGCCGCAGGGAGGG
CTGTGGTGCCGGTGCTTCCAACAAGGACAGCCCTCCTTGACCCTGAAAGGAACACT
GGCTTGAAGGACTGCAGACAGGCTCTGAGGGGCACGCCCTCCTCAGCGAGAGGCAG
CAAGGTGGCCACAGTGTCACTGGTCAGGTGCTTCTCACCACGGGAAAGCCGCCGAC
CTGTGACTCGCTTGAGATGGGAAAGCGGCGCCACAGACCCCGGGTCTCCTTGGCTGT
CTGTGGGCCGCCCCTGGCCACCTTGTCCTGGCTCGCAGGGTGCAGGAGCGCCTCGTT
CTCTGGGTGGCCGGCTTGCTGCTCCGGTTTGGGCTGTCTTACCATAACACCGTCCCA
GGGCTCTGCAGGCCACTGTGAGCGCTGGCTCCCTGGGCAGTGCTCCTCCGTGTGGAC
TGTGCCTCAGGCCAGGGCTCACCAGCTGGGGTCCTGTCCGGAAGGATGGATCTTTCT
GGGAGCTGCGCCGGACAGAGTGGGGAGCTCCTAGTTTGTGGGGGGAAGCTTTGATA
TCCATGCCACGTCCATCCACCCCACCCCTTTTCGTCACGAGCACAATGGTCTTACATT
GGATTTTTGT
SEQ ID NO: 2 (EXTL3_HUMAN Amino Acid Sequence GenBank: AAC39598.1)
MTGYTMLRNGGAGNGGQTCMLRWSNRIRLTWLSFTLFVILVFFPLIAHYYLTTLDEAD
EAGKRIFGPRVGNELCEVKHVLDLCRIRESVSEELLQLEAKRQELNSEIAKLNLKIEACK
KSIENAKQDLLQLKNVISQTEHSYKELMAQNQPKLSLPIRLLPEKDDAGLPPPKATRGCR
LHNCFDYSRCPLTSGFPVYVYDSDQFVFGSYLDPLVKQAFQATARANVYVTENADIAC
LYVILVGEMQEPVVLRPAELEKQLYSLPHWRTDGHNHVIINLSRKSDTQNLLYNVSTGR
AMVAQSTFYTVQYRPGFDLVVSPLVHAMSEPNFMEIPPQVPVKRKYLFTFQGEKIESLR
SSLQEARSFEEEMEGDPPADYDDRIIATLKAVQDSKLDQVLVEFTCKNQPKPSLPTEWA
LCGEREDRLELLKLSTFALIITPGDPRLVISSGCATRLFEALEVGAVPVVLGEQVQLPYQD
MLQWNEAALVVPKPRVTEVHFLLRSLSDSDLLAMRRQGRFLWETYFSTADSIFNTVLA
MIRTRIQIPAAPIREEAAAEIPHRSGKAAGTDPNMADNGDLDLGPVETEPPYASPRYLRN
FTLTVTDFYRSWNCAPGPFHLFPHTPFDPVLPSEAKFLGSGTGFRPIGGGAGGSGKEFQA
ALGGNVPREQFTVVMLTYEREEVLMNSLERLNGLPYLNKVVVVWNSPKLPSEDLLWP
DIGVPIMVVRTEKNSLNNRFLPWNEIETEAILSIDDDAHLRHDEIMFGFRVWREARDRIV
GFPGRYHAWDIPHQSWLYNSNYSCELSMVLTGAAFFHKYYAYLYSYVMPQAIRDMVD
EYINCEDIAMNFLVSHITRKPPIKVTSRWTFRCPGCPQALSHDDSHFHERHKCINFFVKV
YGYMPLLYTQFRVDSVLFKTRLPHDKTKCFKFI

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims.

By way of non-limiting illustration, examples of certain embodiments of the present disclosure are given below.

### EXAMPLES

Traumatic brain injury (TBI) is a leading cause of death and disability worldwide, presenting a wide range of clinical symptoms and outcomes that complicate treatment strategies. Traumatic brain injury (TBI) is an injury that affects brain function, and it is one of the major causes of disability and death in the United States (Traumatic Brain Injury/Concussion I Concussion I Traumatic Brain Injury I CDC Injury Center. 2023, https://www.cdc.gov/traumaticbraininjury/index.html). Despite its high prevalence and public health emergency the underlying pathophysiology is not fully understood. Furthermore, the long-term effect of the association with several types of dementia in later life is the need to better understand the pathophysiological mechanism of TBI (Traumatic Brain Injury/Concussion I Concussion I Traumatic Brain Injury I CDC Injury Center. 2023, https://www.cdc.gov/traumaticbraininjury/index.html; Capizzi A, et al., Med Clin North Am. 2020; 104(2):213-38; DePalma R G, et al., Behav Brain Res. 2018, 15; 340:102-5 (“DePalma, et al.,”)). Alzheimer's disease (AD) is the most common type of dementia and is one of the leading causes of death in the United States. In 2020 more than 5.8 million Americans lived with AD (Kalra S, et al., Inflammopharmacology. 2022; 30(4):1153-66 “Kalra, et al.”; Omalu B. et. al., Prog Neurol Surg. 2014; 28:38-49 (“Omalu, et al.”); Ma X, et al., Mol Neurobiol. 2019; 56(8):5332-45 “Ma, et al.”). The early cellular phase of AD happened with an accumulation of extracellular amyloid beta (Aβ) plaques leading to the spread of Tau pathology and intraneuronal deposits of neurofibrillary tangles (NFTs) (DePalma, et al., Kalra, et al., Omalu, et la.). Aβ accumulation is proposed to initiate AD via damaging synapses and neurons (DePalma, et al.). Some of AD's pathological features such as increase and accumulation in amyloid beta (Ab), and hyperphosphorylated tau (p-Tau) were found in postmortem brains of TBI and CTE (Hyder A A, et al., NeuroRehabilitation. 2007; 22(5):341-53; Traumatic Brain Injury (TBI) I National Institute of Neurological Disorders and Stroke, 2023, https://www.ninds.nih.gov/health-information/disorders/traumatic-brain-injury-tbi). The mechanism by which this occurs is still unclear. Heparan sulfate (HS) is a highly sulfated linear polysaccharide and covalently attaches to protein cores to form HS proteoglycans to express on the cell surface and in the extracellular matrix (Zou Z, et al., J Neuroinflammation. 2021, 14; 18:134; Ramos-Cejudo J, et al., EBioMedicine. 2018 Jan. 31; 28:21-30). It plays crucial roles in modulating cell-cell and cell-matrix interactions and signaling through its interactions with various protein ligands.

This variability underscores the urgent need for a deeper understanding of TBI's pathophysiology to develop more effective therapies. TBI triggers a cascade of neurodegenerative processes, including amyloid β (Aβ) and tau deposition, and cerebrovascular disturbances, which closely mirror Alzheimer's disease (AD) pathology. TBI is increasingly recognized as a significant risk factor for AD. However, the precise molecular mechanisms underlying TBI and their link to TBI-associated AD remain poorly understood. Heparan sulfate (HS), a sulfated glycosaminoglycan on cell surfaces and in the extracellular matrix (ECM), is crucial for cell signaling and interactions. Clinical studies have shown that trauma increases HS shedding from the luminal surface of brain endothelial cells (bEC), raising HS levels in circulation; the associated HS proteoglycan Syndecan-1 (SDC-1) in blood serves as a biomarker of trauma severity. Preliminary studies analyzing single-cell RNA sequencing (scRNA-seq) data from a pediatric TBI mouse model and AD patients have shown reduced expression of HS biosynthesis genes in brain endothelial cells (bECs). The reduction in bEC-HS expression was also observed in the repetitive mild TBI (rTBI) mouse model and the endothelial cell-specific Ext1 haplodeficient mice. The endothelial cell-specific Ext1 haplodeficient mice with human mutant amyloid overexpression 5xFAD genetic background exhibited increased Aβ accumulation in the brain and reduced Aβ uptake by bECs. Meanwhile, in mild rTBI, the endothelial cell-specific Ext1 haplodeficient mice demonstrated greater motor deficits, microvascular damage, and increased Aβ and tau deposition, underscoring the critical role of bEC-HS in TBI and TBI-induced AD pathology. The studies have provided key evidence: 1) bEC-HS is necessary for Aβ clearance in mouse brains, and this process can be enhanced by upregulating bEC-HS expression; 2) Expression of the HS biosynthesis initiation enzyme in bECs is downregulated in a pediatric TBI mouse model; 3) bEC-HS expression is reduced in the repetitive TBI (rTBI) mouse model; and 4) Genetically reducing bEC-HS expression worsens TBI pathology, including cerebrovascular damage and Aβ and tau deposition, highlighting the vital role of bEC-HS in TBI and TBI-induced AD pathology. Based on these findings, reduced bEC-HS expression that disrupts cerebral vascular function was expected, contributing to TBI, and impairs Aβ clearance, thereby promoting AD development.

The studies are expect to reveal significant correlations between changes in HS expression, especially plasma and bEC HS, and TBI-related pathologies, including AD pathology, and demonstrate that reduced bEC-HS exacerbates TBI and associated AD pathology, while increased bEC-HS have protective effects. These findings provide insights into the role of HS in TBI and AD, highlighting bEC-HS as a new therapeutic target for the disease.

Characterize Change in bEC-HIS Expression and Investigate its Correlation With TBI and Associated AD Pathology.

Biochemical analyses and immunostaining can be conduct to assess changes in HS levels and structure in plasma and brain tissues in the mTBI model. scRNA-seq can be used to evaluate dynamic changes in HS gene expression across all major brain cell types, including bECs. Additionally, a detailed pathological analysis can be performed to examine cortical lesions, neuronal injury, neuroinflammation, vascular injury and circulation insufficiency, changes in ECM proteins and basement membrane structures, Aβ and Tau deposition, and motor and memory functions. the correlation between changes in HS expression and these pathological features can be determined.

The rTBI mouse model mirrors the reduction in endothelial HS indicated in trauma patients and aligns with the scRNA-seq data from the reported pediatric mouse TBI study (Rahbar, E. et al. J Transl Med 13, 117, (2015) (“Rahbar, et al.”); Gonzalez Rodriguez, E. et al. J Am Coll Surg 225, 419-427, (2017) (“Gonzalez Rodriguez, et al.”); Richter, R. P. et al. Front Cell Dev Biol 12, 1390794, (2024) (“Richter, et al.”)), representing a well-suited model to study bEC-HS's role in TBI. The correlation of changes in HS expression with TBI pathology in the model can be examined.

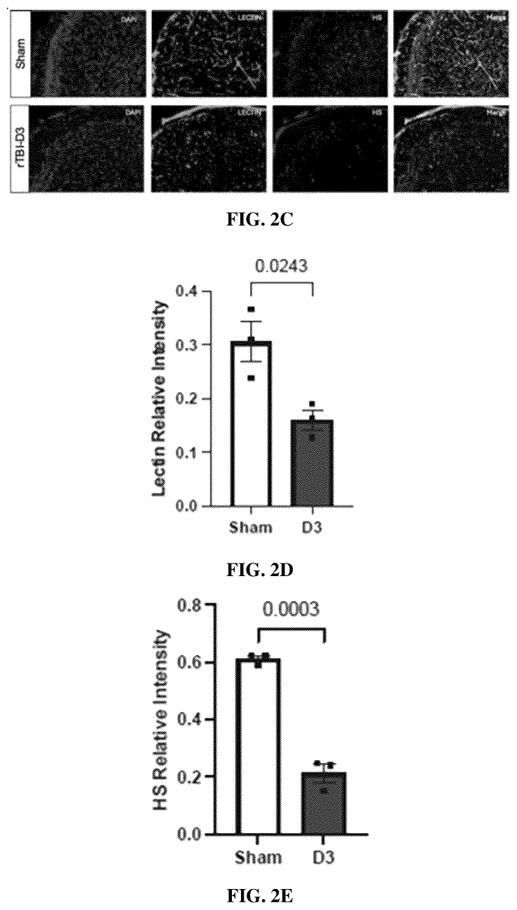

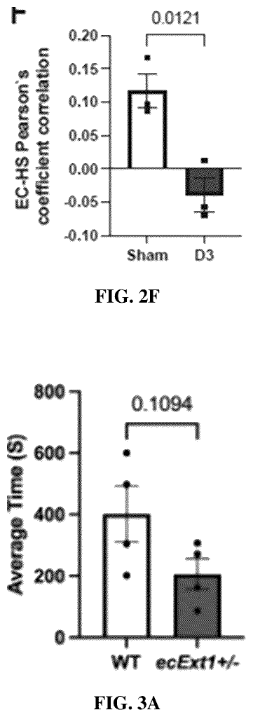

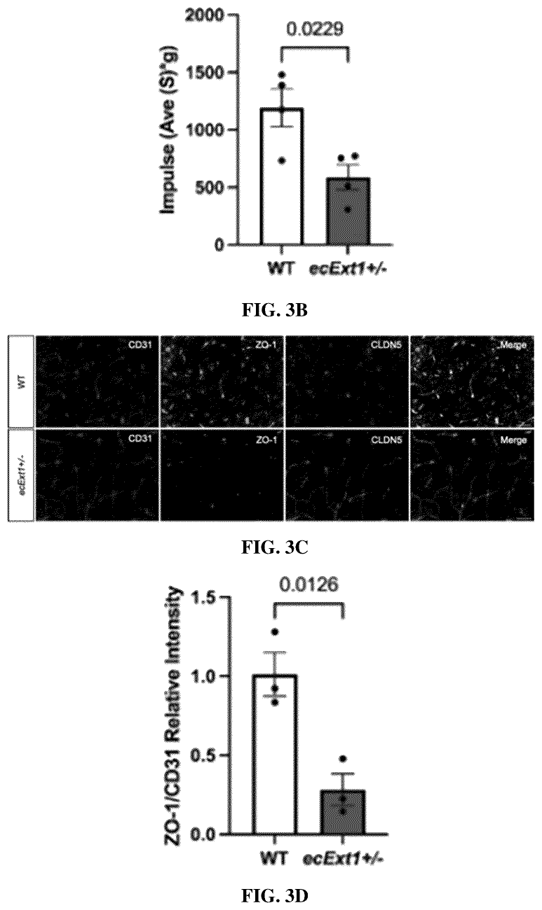

Determine HS Expression—levels, structure, and related gene expression: HS is a linear polysaccharide modified with N-sulfation (NS) and 2-, 3-, and 6-O-sulfation (2S, 3S, 6S) (Bishop, J. R., et al., Nature 446, 1030-1037, (2007) (“Bishop, et al.,”), Li, J. P. et al., Int Rev Cell Mol Biol 325, 215-273, (2016) (“Li, et al.,”)). Its synthesis begins with the enzyme Ext13 and is extended by the Ext1-Ext2 co-polymerase, along with sulfotransferases like Ndsts for NS and Hs3sts for 3S (Table 1). These sulfation patterns create specific binding sites for protein ligands, defining HS's biological functions (Bishop, et al., and Li, et al.). To evaluate HS expression, the model can be assessed before and on Days 1, 3, 8, and 21 post-TBI (FIG. 2A): 1) measure plasma HS levels by ELISA (Richter, et al.) and analyze disaccharide and 3-O-sulfated tetrasaccharides (Wang, L., et al., Nat Immunol 6, 902-910, (2005) (“Wang, 2005”), Zhang, B. et al. J Clin Invest 124, 209-221, (2014) (“Zhang, et al.”), Qiu, H. et al. Nat Methods 15, 889-899, (2018) (“Qiu, et al.”), Wang, Z. et al. Sci Adv 9, eadf6232, (2023) (“Wang, 2023”)). 2) Stain brain tissues to assess HS expression in bECs (CD31+), pericytes (CD13+), neurons (MAP2+), oligodendrocytes (NG2+ or CNPase+), microglia (Iba1+), andastrocytes (ALDH1L-1+), with anti-HS antibody 3G10 (HS chain number), 10E4 (HS level), and phage display antibodies (NS, 2S, 3S, and 6S); and 3) scRNA-seq of brain tissues to identify HS gene expression changes in bECs, and other cell type and analysis of differentially expressed genes to determine associated biological processes and pathways.

TABLE 1
HSPG gene expression changes in bECs in a pediatric TBI mouse model.
The data was extracted from Gene Expression Omnibus GSE95401

| | Expression (FPKM) | | | |
|---|---|---|---|---|
| Gene | Control | TBI | P-value | Function in HS metabolism |
| Extl3 | 256.8 | 183.1 | 0.0084 | Biosynthesis initiation |
| Ext1 | 35.9 | 49.3 | 0.0103 | HS chain polymerization |
| Ndst1 | 57.3 | 75.3 | 0.0107 | N-sulfation |
| Hs3st1 | 28.9 | 17.1 | 0.0014 | 3-O-sulfation |
| HPSE | 0.2 | 0.2 | 0.8010 | HS degradation |
| SDC1 | 3 | 4.6 | 0.0329 | HSPG |

Pathology analysis over the mTBI time course (FIG. 2A): pathological changes in the rTBI model (Wu, Y., et al., Front Physiol 11, 1030, (2020) (“Wu, 2020”), Yao, Y., et al., Nat Commun 5, 3413, (2014) (“Yao, et al.”), Xu, L. et al. Cell Rep 41, 111709, (2022) (“Xu, et al.”), Kang, M. et al., Cell Rep 43, 114123, (2024) (“Kang, et al.”)) can be systematically assess. I) General pathology includes cortical lesions by cresyl violet staining, cerebral edema by weighing brain water content, neuronal apoptosis by counting TuneL⁺-NeuN⁺ cells, axonal injury by SMI-32 antibody staining (Libbey, J. E., et al., Discov Med 18, 79-89 (2014) (“Libbey, et al.”)) and Western blotting for synapsin I, microgliosis, and astrogliosis. 2) Cerebrovascular damage: Microvascular injury and BBB dysfunction using immunostaining for plasma-derived IgG, extravascular fibrin deposits, and extravasation of 555-cadaverine, pericyte (CD13+) coverage along bECs. ECM proteins and basement membrane structures by staining for collagen IV and laminins, and microcirculation measured using the Laser Speckle Contrast Imaging. 3) AD pathology: immunostaining for Aβ and Tau deposition. 4) Motor function: rotarod and hanging wire tests; 5) Memory and learning: Morris Water Maze and Fear conditioning tests on Days 8 and 21 post-TBI.

Correlations between changes in HS expression are expected including increased plasma HS, reduced bEC-HS, and alterations in HS structure and gene expression and TBI-related pathologies, including neuronal degeneration, cerebrovascular damage, neuroinflammation, increased Aβ and Tau deposition, and behavior deficits. Alternatively, 1) HS antibody staining of some brain cell types, such as microglia, can be challenging due to their ramified processes. To address this, a microglia reporter mouse can be used, such as TMEM119-GFP mice (Kaiser, T. et al., eNeuro 6, (2019) (“Kaiser, et al.”)), stain the isolated TMEM119-GFP+ cells, and perform flow cytometry analysis. 2) scRNA-seq data analysis,

### Results

Establishing an rTBI mouse model which shows reduced bEC and HS staining. Clinical patient study and mouse model scRNA-Seq both indicate a reduction in bEC-HS in TBI (Table 1) (Rahbar, et al.; Gonzalez Rodriguez, et al). To recapitulate this, mild rTBI was chosen, which reflects most TBI cases (Alzheimer's disease facts and figures. Alzheimers Dement 20, 3708-3821, (2024)). The model was refined by adjusting impact times and injury severity. The optimized rTBI model (FIG. 2A) showed transient motor impairment on Day 1 (D1) post-rTBI in the rotarod test (FIG. 2B), with near recovery by D3. The rTBI mice exhibited normal overall brain and vasculature length and branching (data not shown) but showed reduced staining of the endothelial cell marker tomato-lectin and HS, including bEC-HS, as indicated by decreased HS-lectin correlation (FIG. 2C-2F). This finding mirrors the reduction in endothelial HS indicated in trauma patients and aligns with the scRNA-seq data from the reported pediatric mouse TBI study.

Determine the Role of bEC-HIS in TBI and Associated AD Pathology.

Endothelial-specific HS haplodeficient and overexpression mice can be examined to assess the impact of reduced and increased bEC-HS on TBI and associated AD pathology. Correlations between changes in HS expression are expected, particularly in plasma and bECs, and TBI pathologies, including AD, and demonstrate that reduced bEC-HS exacerbates, while increased HS alleviates, TBI and associated AD pathology. This study is expected to significantly advance the understanding of the molecular mechanisms underlying TBI and linking TBI to AD and identify bEC-HS as a potential therapeutic target, aligning with the AHA's mission to improve cardiovascular and neurovascular health, Reduced bEC-HS exacerbates motor function deficits, disrupts BBB tight junction protein expression, and worsens Aβ and Tau deposition in rTBI.

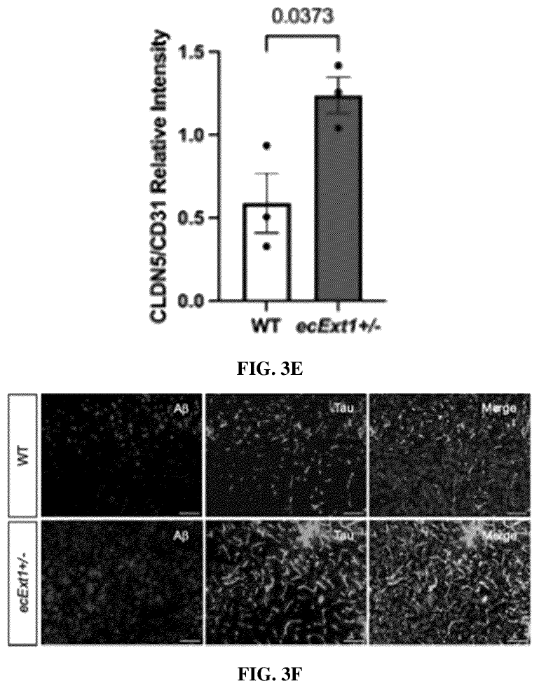

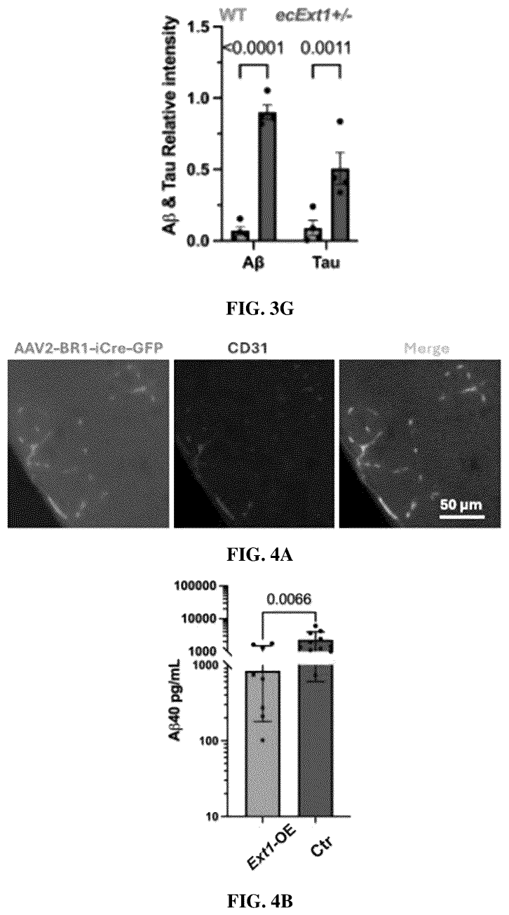

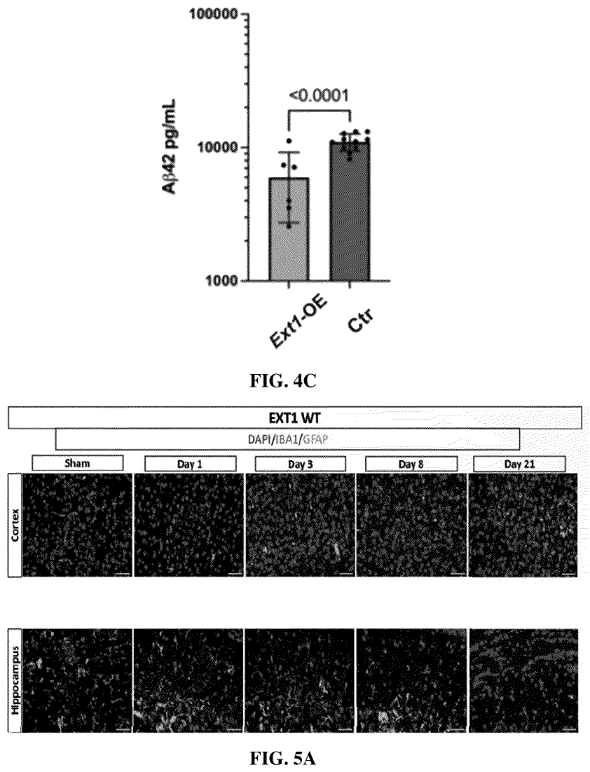

Roles of reducing and increasing bEC-HS in TBI pathology over time (FIG. 2A); the impact of reduced bEC-HS on TBI and related AD pathology using the ecExt1^{+/-} mice was investigate. To evaluate the effects of increased bEC-HS, bEC-targeted AAV2-BR/vector was utilized to specifically overexpress Ext1 in bECs in mice (Korbelin, J. et al. EMBO Mol Med 8, 609-625, (2016) (“Korbelin, et al.”)). The AAV2-BR1 vector has shown high specificity and long-lasting expression in bECs following intravenous delivery, even in adult mice (Korbelin, et al.). It has successfully restored vascular function in incontinentia pigmenti mice following AAV-BR1-NEMO injection (Korbelin, et al.) 3czxv the AAV2-BR1-iCre-GFP vector was tested via intracranial injection and observed 100% overlap with CD31, confirming its high specificity and efficiency for gene expression in bECs (FIG. 4A). Additionally, AAV2-BR1-iCre-Ext1 i.v. injection reduced insoluble Aβ40 and Aβ42 in 5xFAD mice (FIG. 4B-4C), indicating that increased bEC-HS enhances Aβ clearance in the brain. the AAV2-BR1-iCre-Ext1 vector can be administered to wild-type mice pre-TBI, during rTBI (after the 2nd impact), and on Day 1 post-TBI. bEC-HS expression can be assessed by quantifying Ext mRNA in isolated bECs and measuring cell surface HS levels by 10E4 antibody staining and flow cytometry analysis. Finally, it can be determined whether AAV2-BR1-iCre-Ext1 expression mitigates TBI and related AD pathology. It is anticipated that decreasing bEC-HS exacerbates while increased bEC-HS ameliorates TBI and related AD pathology. The altered BBB-related tight junction protein expression might reduce the efficiency of AAV2-BR1-iCre-Ext1 expression in bECs in rTBI mice. If this occurs, the vector dose can increase to match Ext1 expression with the sham group.

TBI manifests cerebrovascular injury and is the most significant environmental risk factor for AD. TBI is a leading cause of death and disability globally, with a wide range of clinical symptoms and outcomes that complicate treatment strategies (Galgano, M. et al., Cell Transplant 26, 1118-1130, (2017) (“Galgano, et al.”), Alzheimer's disease facts and figures. Alzheimers Dement 20, 3708-3821, (2024)). This variability underscores the urgent need for a deeper understanding of TBI's pathophysiology to develop more effective therapies. TBI triggers a range of neurodegenerative processes, including axonal and dendritic damage, excitotoxicity, neuroinflammation, and neuronal cell death (Blennow, K., et al., Neuron 76, 886-899, (2012) (“Blennow, et al.”)). It also results in cerebrovascular complications such as edema, impaired circulation, and blood-brain barrier (BBB) disruption (Wu, Y., et al., Front Physiol 11, 1030, (2020) (“Wu, 2020”), Furuya, Y., et al., Neurosurgery 52, 340-345; discussion 345-346, (2003) (“Furuya, et al.”), Menon, D. K. Curr Opin Crit Care 12, 85-89, (2006) (“Menon, et al.”), Wu, Y. et al. Acta Neuropathol Commun 9, 74, (2021) (“Wu, 2021”)), which resemble AD pathology (Sandsmark, D. K., Neuron 103, 367-379, (2019)). TBI exacerbates AD-related events, such as β-amyloid (Aβ) accumulation and the formation of neurofibrillary tangles with hyperphosphorylated tau protein (Yu, F., et al., J Neurotrauma 29, 2342-2351, (2012), Scott, G. et al. Neurology 86, 821-828, (2016)). TBI is considered the most significant environmental risk factor for AD2,11. However, the molecular mechanisms driving TBI and its connection to AD development remain poorly understood (Galgano, et al., Kenney, K. et al., JAMA Neurol 75, 1043-1044, (2018)).

HS expression is dysregulated in TBI, but its roles remain unclear. HS is a highly sulfated linear polysaccharide that forms HS proteoglycans (HSPGs) by attaching to protein cores. HSPGs are found on cell surfaces, such as syndecans and glypicans, and within the ECM, including agrin, perlecan, and collagen XVIII (Bishop, et al., and Li, et al.). Through HS chains, HSPGs interact with protein ligands, affecting cell-cell and cell-matrix interactions and signaling pathways. During trauma, HS is shed from endothelial cells (EC) and circulating Syndecan-1 (SDC1) serves as a biomarker for TBI severity (Yilmaz, O., et al., Clin Kidney J 12, 611-619, (2019) (“Yilmaz, et al.”), Rahbar, et al., Gonzalez Rodriguez, et al., Naumann, D. N. et al. Shock 49, 420-428, (2018), Richter, R. P. et al. Ann Surg Open 3, (2022) (“Richter, et al. 2022”), Richter, et al.). scRNA-seq from a pediatric TBI mouse model (Munji, R. N. et al. Nat Neurosci 22, 1892-1902, (2019)) reveals downregulation of Ext13, a key HS biosynthesis gene, in bECs during acute TBI, indicating reduced bEC-HS expression despite upregulation of downstream HS biosynthesis genes like Ext1 and Ndst1 (Table 1). Heparanase (HPSE), the enzyme responsible for shedding HS (Yilmaz, et al., Rahbar, et al., Richter, et al. 2022), remains unchanged, while SDC1 expression is upregulated, possibly as a compensatory response. No studies have systematically examined changes in HS expression in bECs during TBI or its role in TBI pathology.

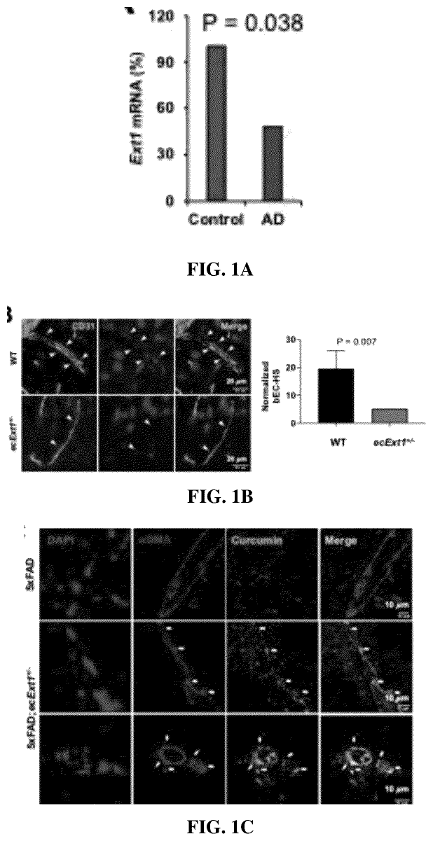

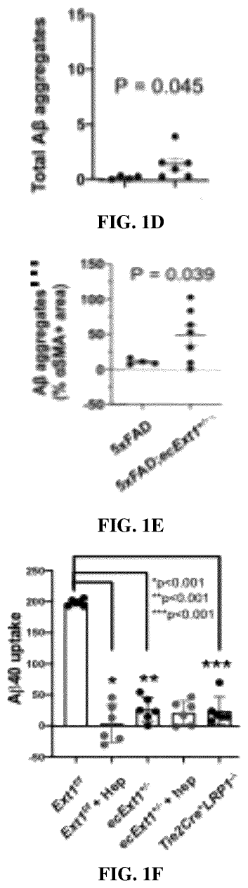

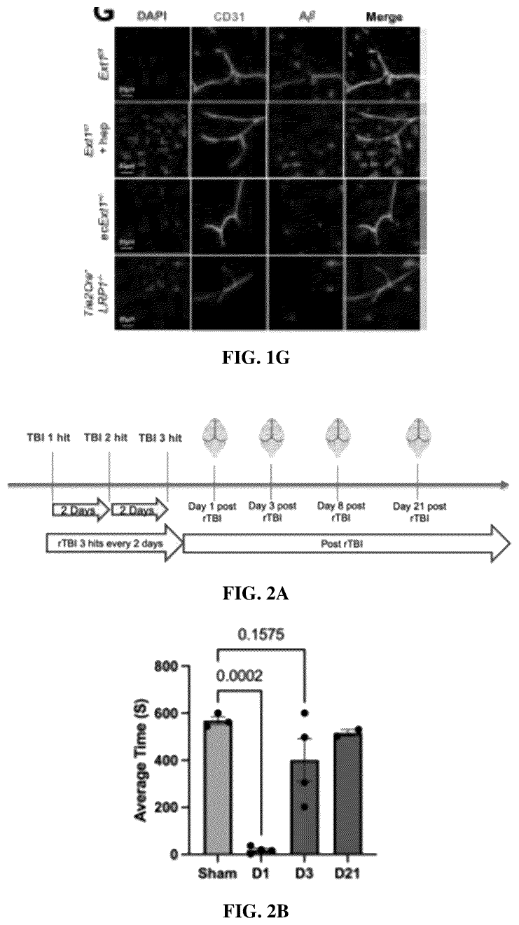

Reduced bEC-HS expression may contribute to TBI-induced AD pathology. AD is a progressive neurodegenerative disorder marked by early neurovascular damage and the accumulation of misfolded Aβ and tau in the brain, with its molecular mechanisms largely unknown (Jack, C. R., Jr. et al. Alzheimers Dement 14, 535-562, (2018)). In AD, HS co-deposits with Aβ plaques in the brain parenchyma and cerebral vasculature (Shimizu, H., et al., J Clin Neurosci 16, 277-282, (2009), Huynh, M. B. et al. PLoS One 14, e0209573, (2019), Hosono-Fukao, T. et al. J Neurosci Res 89, 1840-1848, (2011)). Analysis of scRNA-seq data from AD patients (Mathys, H. et al. SNature 570, 332-337, (2019)) revealed a 52% decrease in expression of Ext1, an enzyme essential for HS chain polymerization (Table 1), in bECs (FIG. 1A), while other brain cell types showed no change. To model this downregulation, tamoxifen-inducible endothelial-specific Ext1 haplodeficient mice (Cdh5(PAC)-CreERT2; Ext1f/+, ecExt1 +/−) was generated which exhibit approximately 65% reduced bEC-HS expression (FIG. 1B) and appear normal, consistent with reported Ext1 heterozygous mice (Zak, B. M. et al. Bone 48, 979-987, (2011), Mooij, H. L. et al. J Lipid Res 56, 665-673, (2015)). Notably, ecExt1 +/− mice show increased Aβ deposition with a 5xFAD AD mouse background (FIG. 1C-1E) and reduced Aβ uptake by bECs (FIG. 1F, 1G). These findings underscore the critical role of bEC-HS in Aβ clearance and suggest that reduced bEC-HS may contribute to TBI-induced AD pathology.

It is expected to identify correlations between changes in HS expression, particularly in plasma and bECs, and TBI pathologies, including AD, and to demonstrate that reduced bEC-HS exacerbates while increased HS alleviates TBI and associated AD pathology. This study can advance understanding of the molecular mechanisms underlying TBI and linking TBI to AD and identify bEC-HS as a potential therapeutic target.

### Results

Reduced bEC-HS expression exacerbates motor deficit, BBB damage, and Aβ and Tau deposition in rTBI. The ecExt1^{+/-} mice maintained normal BBB permeability, as assessed by i.v. injected FITC-dextran (4 kDa) (data not shown). However, these mice exhibited more severe motor coordination deficits on D3 post-TBI in the hanging wire test and a greater tendency of impairment in the rotarod test (FIG. 3A, 3B). They also showed decreased expression of the BBB-associated protein ZO-1 and increased expression of CLDN5 (FIG. 3C-3E), No differences were observed in vascular length, branching, pericyte coverage, or AQP4 expression (data not shown). Additionally, ecExt1^{+/-} mice demonstrated increased deposition of Aβ and tau (FIG. 3F, 3G). These results indicate that reduced bEC-HS exacerbates TBI and associated Alzheimer's disease pathology.

Statistical analyses, experimental rigor, and biological variables. The number of mice is determined by power calculations based on preliminary results with a power of 0.85, using a slightly more conservative effect size of 1.5×. Mouse genotype and treatment can be blinded to technicians collecting the data. Data can be collected from at least three independent experiments, combined, and analyzed for statistical significance. Data points can only be excluded if there is a clear experimental issue; otherwise, the entire experiment can be repeated. Since TBI outcomes (Teterina, A. et al. Sci Rep 13, 18453, (2023) (“Teterina, et al.”)) and AD incidence (Vina, J. et al., J Alzheimers Dis 20 Suppl 2, S527-533, (2010) (“Vina, et al.”)) vary by sex, both male and female mice can be included in the study. Sex can be considered a biological variable in the statistical analyses.

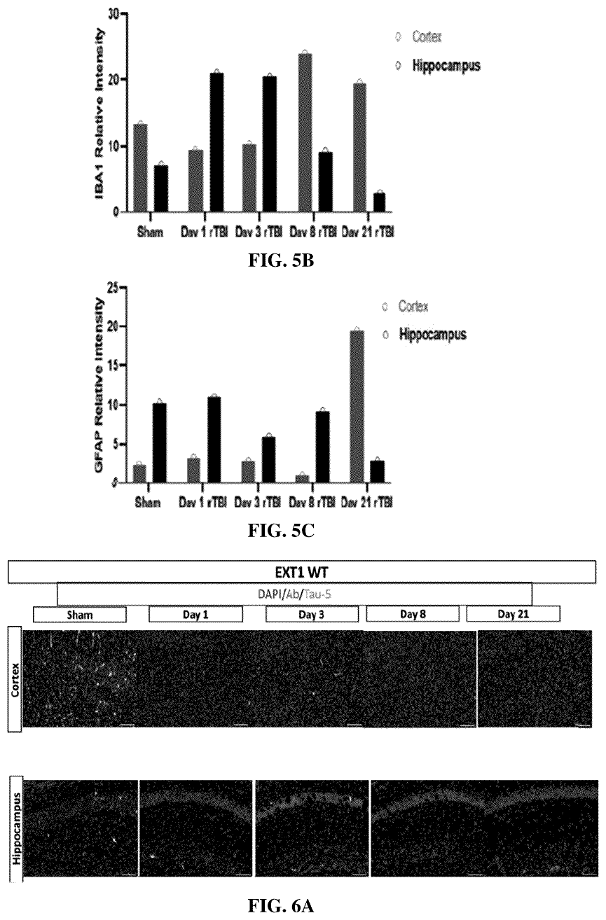

Inflammation during TBI: After TBI, the brain blood vessel is disrupted causing migration of inflammatory cells such as leukocytes, and filtration of vascular proteins to the neuronal space all of which lead to the activation of microglia and astrocytes. This can lead to inflammation could spread and damage the central nervous system as well as further development of secondary diseases such AD. There is more inflammation on day 8 and day 21 in the cortex for microglial markers. See FIGS. 5A-5C.

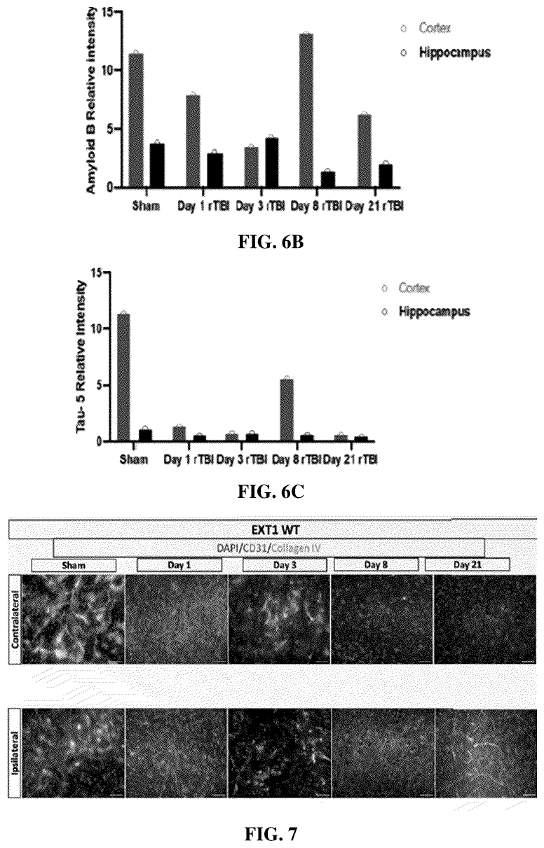

Alzheimer's Disease Pathology in TBI: After TBI there is an accumulation in amyloid beta (Aβ) and hyperphosphorylated tau (P-Tau) deposits. Vascular shear stress occurring during TBI can disrupt BBB as well as HS present in the capillaries and contribute to Aβ accumulation. Aβ clearance from the brain is through enzymatic pathways or non-enzymatic pathways which include uptake by microglial phagocytosis. After TBI, there is an accumulation of Ab and Tau on day 1, day 8, and day 21. See FIGS. 6A-6C.

Change in the capillaries after TBI: One of the characteristics of TBI is change in the brain capillaries. In fact, the rush of blood flow enlarge the brain blood vessels even leading to the capillary's disruption. See FIG. 7.

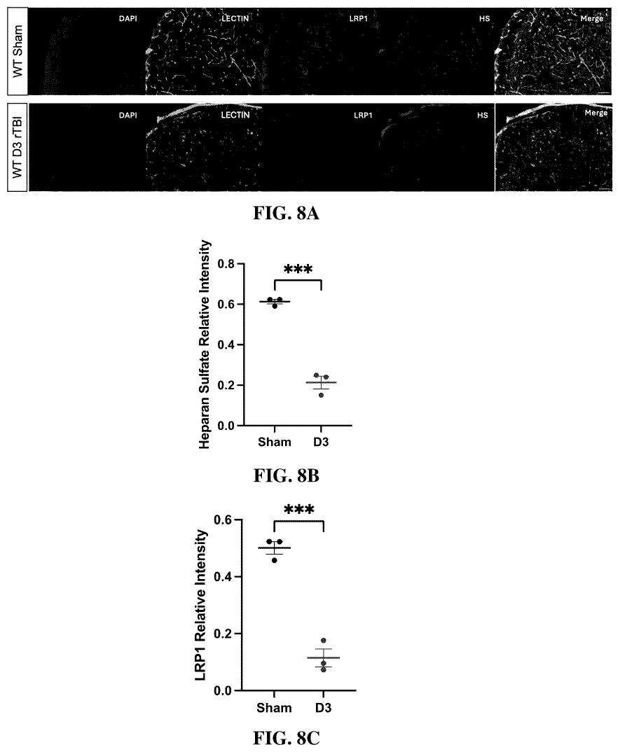

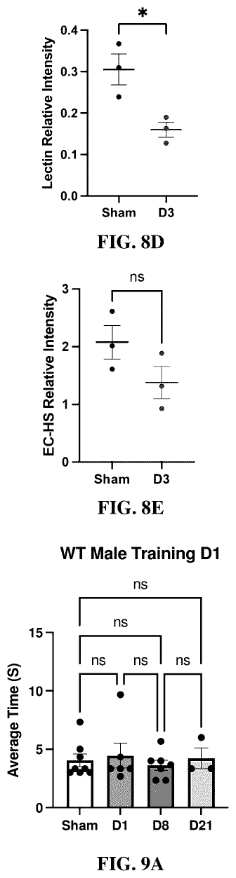

HS and LRP1 expression in WT mice compared to sham 3 days after rTBI. The expression of heparan sulfate (HS) and low-density lipoprotein receptor-related protein 1 (LRP1) was evaluated on control WT mice comparing sham to day 3 after rTBI and reported significant decrease in the brain. See FIGS. 8A-8E.

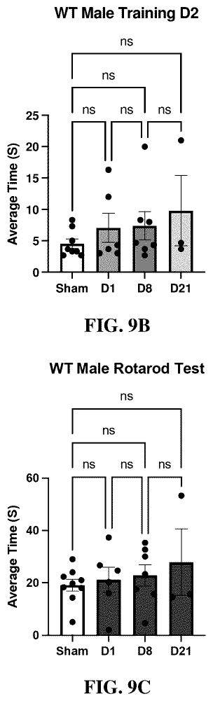

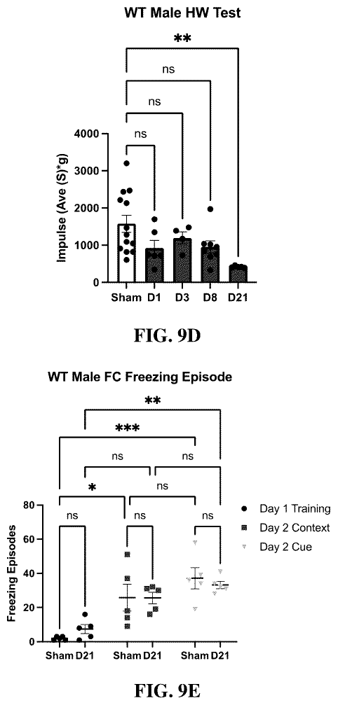

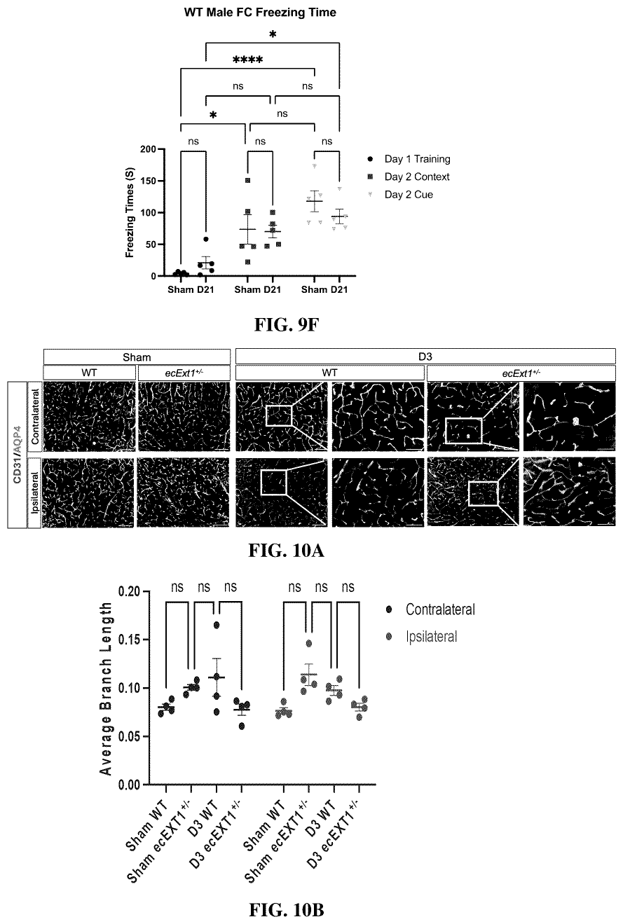

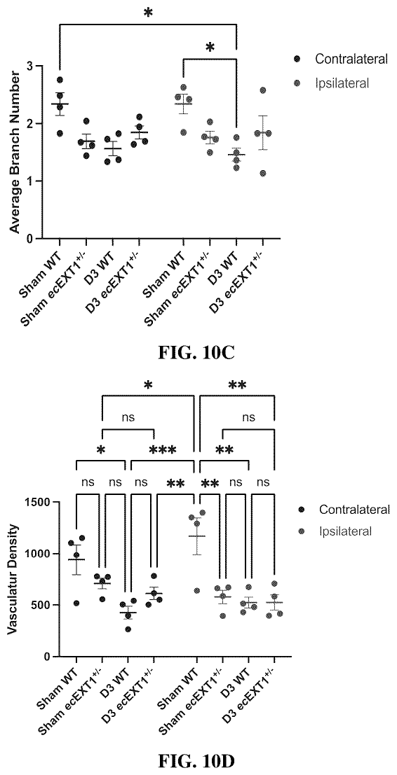

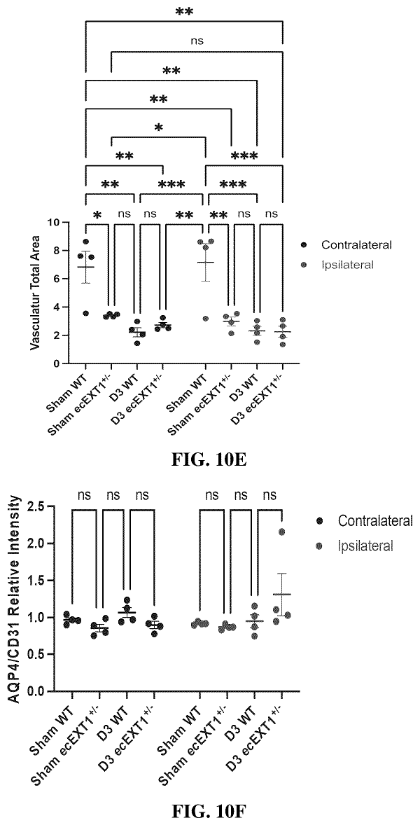

Motor, coordination, and learning ability of WT mice compared to sham after rTBI. Motor and coordination was evaluated as well as learning ability of mice on control WT mice comparing sham to day 1, 8, 3 and 21 after rTBI change in behavior of the mice after rTBI. See FIGS. 9A-9F.

Vasculature structure comparing WT and ecEXT1 +/− mice. Vasculature structure was assessed comparing WT and ecEXT1 +/− mice where HS expression was downregulated. We compared sham to day 3 after rTBI and reported change in the brain vasculature.

TBI is one of the leading causes of death and disability worldwide. It has a long-term effect associated with several types of dementia in later life such as AD. TBI mechanism involves disruption of the blood-brain barrier (BBB) and the secondary injury observed in TBI involves inflammation, oxidative stress, and even an increase in amyloid beta (Aβ), hyperphosphorylated tau. The risk of developing AD increases after TBI as TBI characteristics and AD are similar. Heparan sulfate present in every cell has a protective effect, particularly in the capillaries enhancing its importance and protective effect after TBI.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

What is claimed is:

1. A method for treating or preventing conditions, diseases, or disorders related to, associated with, or caused by traumatic brain injury in a subject, the method comprising:
administering a therapeutically effective amount of a heparan sulfate proteoglycan modifier agent to a subject.

2. The method of claim 1, wherein the method increases expression of heparan sulfate proteoglycan in brain endothelial cells compared to the expression of heparan sulfate proteoglycan without administration of an effective amount of heparan sulfate proteoglycan modifier agent.

3. The method of claim 1, wherein the method increases concentration of heparan sulfate proteoglycan in brain endothelial cells compared to the concentration of heparan sulfate proteoglycan without administration of an effective amount of heparan sulfate proteoglycan modifier agent.

4. The method of claim 1, wherein the method increases the concentration of exostosin like glycosyltransferase 3 (EXTL3) in brain endothelial cells compared to the concentration of exostosin like glycosyltransferase 3 (EXTL3) without administration of an effective amount of heparan sulfate proteoglycan modifier agent.

5. The method of claim 1, wherein the heparan sulfate proteoglycan modifier agent is a nucleic acid, a protein/peptide, a small molecule, or any combination thereof.

6. The method of claim 5, wherein the heparan sulfate proteoglycan modifier agent is a nucleic acid.

7. The method of claim 6, wherein the nucleic acid comprises double stranded DNA, single-stranded DNA, complexed DNA, encapsulated DNA, naked RNA, encapsulated RNA, messenger RNA (mRNA), tRNA, short interfering RNA (siRNA), double stranded RNA (dsRNA), micro-RNA (miRNA), a long non-coding RNAs (IncRNAs), antisense RNA (asRNA), self-amplify mRNA (saRNA), guide RNA (gRNA), cRNA, or any combination thereof.

8. The method of claim 6, wherein the heparan sulfate proteoglycan modifier agent comprises a nucleic acid having at least 80% sequence identity to SEQ ID NO: 1 encoding for exostosin like glycosyltransferase 3 (EXTL3), a fragment, or variant thereof having at least 80% sequence identity to SEQ ID NO: 2.

9. The method of claim 6, wherein the nucleic acid encodes for exostosin like glycosyltransferase 3 (EXTL3), a fragment, or variant thereof having at least 80% sequence identity to SEQ ID NO: 2.

10. The method of claim 6, wherein the nucleic acid is a messenger RNA (mRNA).

11. The method of claim 5, wherein the heparan sulfate proteoglycan modifier agent is a protein/peptide.

12. The method of claim 11, wherein the protein/peptide has at least 80% sequence identity to SEQ ID NO: 2.

13. The method of claim 1, wherein the condition, disease, or disorder related to, associated with, or caused by traumatic brain injury is a neurodegenerative disease.

14. The method of claim 13, wherein the neurodegenerative disease is selected from Alzheimer's disease (AD), frontotemporal dementia (FTD), progressive supranuclear palsy, corticobasal degeneration (CBD), and chronic traumatic encephalopathy (CTE).

15. The method of claim 1, wherein the method reduces Beta-amyloid deposition, tau deposition, or any combination thereof of a subject in need thereof.

16. The method of claim 1, further comprising administering the heparan sulfate proteoglycan modifier agent and a pharmaceutically acceptable carrier.

17. The method of claim 1, wherein administration comprises topical, intravenous, subcutaneous, transcutaneous, transdermal, intramuscular, intradermal, intra-arteriole, intralesional, or any combination thereof.

18. A method of reducing Beta-amyloid deposition, tau deposition, or any combination thereof in a subject's brain, the method comprising:
administering a therapeutically effective amount of a heparan sulfate proteoglycan modifier agent to the subject.

19. A method of treating or preventing a neurodegenerative disease in a subject, the method comprising:
administering a therapeutically effective amount of a heparan sulfate proteoglycan modifier agent to the subject.

20. The method of claim 19, wherein the neurodegenerative disease is selected from Alzheimer's disease (AD), frontotemporal dementia (FTD), progressive supranuclear palsy, corticobasal degeneration (CBD), and chronic traumatic encephalopathy (CTE).


SEQUENCE LISTING (2 sequences)

SEQ ID NO: 1 - DNA, 6147 bp, Homo sapiens
  source 1..6147: mol_type = genomic DNA
gcggcggcggcgggtccctgagctggaagccggagagcaagccctggaggttcactctttcaagaagtcgtgtgctgaggtgtaatgctacacaagtcagaggaaggaagggtcctgaaacacatggcctgattgttggcaaaggcatcataagaagctggcatttatttctgttctaacctattactgtataactgtgaatagacactatgcatatttgttggtcagcaaaaccaagaaacaagagctatggcatttgaaaaagtctgtctgattccagggtgtttttcctgggtttcatcatcaggtacctcctccctttcatctcagcaagaatgtggcaccttttatcgtttgataaagattaaggacatgttctttggtcaacagccagaacttaaaatctgctggaatagggtcagagaccatttcagctgcagctgaggaaaatgaaatgttcattttatttggtgccttgtctggggagcacactaactcttctggaaacgtgtcagtgaaacagagatcgttttgtggaatagcaacccatggttatggcgagtgacccgacgtgatctggggggcaggctgcagaggactcatgacaggctataccatgctgcggaatgggggcgcggggaacggaggtcagacctgcatgctgcgctggtccaaccgcatccgcctcacgtggctcagcttcacgctctttgtcatcctggtcttcttcccgctcatcgcccactattacctcaccactctggatgaggctgatgaggcaggcaagcggatttttggtccccgggtggggaacgagctgtgcgaggtgaagcacgtgctggatctgtgccgcatccgggagtcggtgagtgaagagctcctgcagctggaggccaagcgccaagagctgaacagcgagatcgccaagctgaatctgaagatcgaagcctgtaagaagagcattgagaacgccaagcaggacctgctccagctcaagaatgtcatcagccagaccgagcattcctacaaggagctcatggcccagaaccagcccaagctgtccctgcccatccgactgctcccagagaaggacgatgccggcctccctcccccgaaggccactcggggctgccggctacacaactgctttgattattctcgttgccctctcacctctggcttcccggtctacgtctatgacagtgaccagtttgtctttggcagctacctggatcccttggtcaagcaggcttttcaggcgacagcacgagctaacgtttatgttacagaaaatgcagacatcgcctgcctttacgtgatactagtgggagagatgcaggagccggtggtgctgcggcctgctgagctggagaagcagttgtattccctgccacactggcggacggatggacacaaccatgtcatcatcaatctgtcacgtaagtcagatacacagaaccttctctataacgtcagtactggccgtgccatggtggcccagtccaccttctacactgtccagtacagacctggctttgacttggtcgtatcaccgctggtccatgccatgtctgagcccaacttcatggaaatcccaccacaggtgccggtgaagcggaaatatctcttcaccttccagggcgagaagattgagtctctgaggtctagccttcaggaggcccgctccttcgaagaggaaatggagggcgaccctcccgccgactacgatgaccggatcattgccaccctgaaggcggtgcaggacagcaagctggatcaggtcctggtggaattcacctgcaaaaaccagcccaaacccagcctgccgactgagtgggcactgtgtggagagcgggaggaccgcttggaattgctgaagctctccaccttcgccctcatcattacccccggggaccctcgcttggttatttcctctgggtgtgcaacacggctcttcgaagccctggaagtcggtgccgtcccggtggtgctgggggagcaggtccagcttccctaccaggacatgctgcagtggaacgaggcggccctggtggtgccaaagcctcgtgttaccgaggttcatttcctgctcagaagcctctccgatagtgacctcctggctatgaggcggcaaggccgctttctctgggagacttacttctccactgctgacagtatttttaataccgtgctggctatgattaggactcgcatccagatcccagccgctcccatccgggaagaggcggcagctgagatcccccaccgttcaggcaaggcggctggaactgaccccaacatggctgacaacggggacctggacctggggccagtggagacggagccgccctacgcctcacccagatacctccgcaatttcactctgactgtcactgacttttaccgcagctggaactgtgctccagggcctttccatcttttcccccacactccctttgaccctgtgttgccctcagaggccaaattcttgggctcagggactggctttcggcctattggtggtggagctgggggttctggcaaggaatttcaggcagcgcttggaggcaatgttccccgagagcagttcacggtggtgatgttgacttatgagcgggaggaagtgcttatgaactctttagagaggctgaatggcctcccttacctgaacaaggtcgtggtggtgtggaattctcccaagctgccatcagaggaccttctgtggcctgacattggcgtccccatcatggtggtccgtactgagaagaacagtttgaacaaccgattcttaccctggaatgaaattgagacagaggccatcctgtccattgatgacgatgctcacctccgccatgacgaaatcatgtttgggttccgggtgtggagagaagctcgggaccgcatcgtgggcttccctggccgttaccacgcatgggacatcccccatcagtcctggctctacaactccaactactcctgtgagctgtccatggtgctgacaggtgctgccttctttcacaagtattatgcctacctgtattcttatgtgatgccccaggccatccgggacatggtggatgaatacatcaactgtgaggacattgccatgaacttccttgtctcccacatcactcggaagccccccatcaaggtgacctcacggtggacattccgatgcccaggatgccctcaggccctgtctcatgatgactcccacttccacgagcggcacaagtgcatcaacttcttcgtgaaggtgtacggctacatgcccctcctgtacacgcagttcagggtggattctgtgctcttcaagacacgcctgccccatgacaagaccaagtgcttcaagttcatctaggggcagcgcacggtctggggaagaggatgagcagagggaggaagatggctcccaaggttcctaggcattgcaggaccttgggcacatctgctggtgggtggcccagagcctctgctggaaggggcagcaggaggagtggaaggaaaccgctgcctttatcttgaagtcagccacactgggcctggagccctgggcggagtccccggggttccccacacagggcactgactgatagcttacactgaggactgtggcgactctgcagagtcactcacaccgttcgtacgcccaggacagctggttcgtggtttttacattcaataacaactattatgattatttaaaaagagaaagtttcagatttgccattcaaggcttatttatatatatgtgtgtgtatataaatacatgcacacacttgcatacatatatatttttggctgggggagtgtgagttttgcctttctaagggagggaccgcgcaggctcctttgttctgtattctggcggagatgggtcctggccttgtgtcactggcttatccttaaagatcatctcccatcctccccagcgccatctgtgtgcagcaaccagaaagggatgaacttggccctcttgcgggcctggacaaggtctcttccttaccctttctgttgccagtcagcaacctgtaactcacattctcttcccagtgaatccctgggagcgcctgaccctggtgggctgttcagcttcctgctgctggggccagcgatttttgaggatttatctttaggccaggcttgcctccgtacttatccctgctctcccatttctctcttgtttgagagagaatgaggaagcaaagagtgagaaagaataggggctgaagacgccactcccagatggctctttctatcctgctcttctgttgaaacacacgtgctgtgggcctcaggcgtttctgaagtgctctttcttggattggacaggagatcagcagcgtgcacatctgctgtggtctgaagtggtttgcaggtcagcctcctctccctagtgtagagcaagccagtgtccttcgaggaacccacccggctggccgggaagttttacagcaaggcgcctgccttgggataattccttggtgaaattcaccttccccccgcctctgtctggagccccatcctgtgttatctgtggtttttggacccctaatgtcagcttggctgtaggactccccgaggtttggtatgtgctagaacaatgggaggctgtgatttgctgtgtaagctcacatccagccttggaatctaacgggcattcacaacccgagttaccactttccactccctgcttaggattctgttccctgggctgaaactgaaataagctaattttttgggtcacggtggcagtaggggaacctaggagggtgtgagtggcatttgtcagggatttagcccatgacgtgtttcttgaaccctactttctggaagtggagttgactctggaagttttctagcaactgaacaaaagctcaggtttgtcctggtcatgcacatgccttaagccagttccgtcttccctagaccttggcatcctgtgcttctatttcttggaatacgttctcctctgacctgcctgtaccacgtgggtcctcttcaagtactgttttgaagctgggctcttttgtgtagctcccacccacctgtagggctagctcggcttaagggaactctccccattggcaaaccggacccggccgccgccaggactgtgtttccaaaggttccccgcccccaaccccagcatcagcctgtagctcccctgctgaggcagtgtggttatgttcccagcagtgggggtcagacgcccttcctcagaactttctagttgccctctacctgactcctgacttgtattccttttagcagtagccttcttccctcggggagccaaagagtgtggtgtgtggcgctatattgtggctgctatttcatctggtttcttttaatgtgaggaactcacatactgacttcagtgggactcggtgagccggggccgtctgtgtggtgggaccccctttagcgggactcagtgagctggggccgtctgtgtggtggagccagggcctctccctttagtggagccaggttgtcgggccccgaatgtcactggtggatctaagaagggctgagtggtctgacaccaaaacatgccgcagggagggctgtggtgccggtgcttccaacaaggacagccctccttgaccctgaaaggaacactggcttgaaggactgcagacaggctctgaggggcacgccctcctcagcgagaggcagcaaggtggccacagtgtcactggtcaggtgcttctcaccacgggaaagccgccgacctgtgactcgcttgagatgggaaagcggcgccacagaccccgggtctccttggctgtctgtgggccgcccctggccaccttgtcctggctcgcagggtgcaggagcgcctcgttctctgggtggccggcttgctgctccggtttgggctgtcttaccataacaccgtcccagggctctgcaggccactgtgagcgctggctccctgggcagtgctcctccgtgtggactgtgcctcaggccagggctcaccagctggggtcctgtccggaaggatggatctttctgggagctgcgccggacagagtggggagctcctagtttgtggggggaagctttgatatccatgccacgtccatccaccccaccccttttcgtcacgagcacaatggtcttacattggatttttgt

SEQ ID NO: 2 - protein, 919 aa, Homo sapiens
MTGYTMLRNGGAGNGGQTCMLRWSNRIRLTWLSFTLFVILVFFPLIAHYYLTTLDEADEAGKRIFGPRVGNELCEVKHVLDLCRIRESVSEELLQLEAKRQELNSEIAKLNLKIEACKKSIENAKQDLLQLKNVISQTEHSYKELMAQNQPKLSLPIRLLPEKDDAGLPPPKATRGCRLHNCFDYSRCPLTSGFPVYVYDSDQFVFGSYLDPLVKQAFQATARANVYVTENADIACLYVILVGEMQEPVVLRPAELEKQLYSLPHWRTDGHNHVIINLSRKSDTQNLLYNVSTGRAMVAQSTFYTVQYRPGFDLVVSPLVHAMSEPNFMEIPPQVPVKRKYLFTFQGEKIESLRSSLQEARSFEEEMEGDPPADYDDRIIATLKAVQDSKLDQVLVEFTCKNQPKPSLPTEWALCGEREDRLELLKLSTFALIITPGDPRLVISSGCATRLFEALEVGAVPVVLGEQVQLPYQDMLQWNEAALVVPKPRVTEVHFLLRSLSDSDLLAMRRQGRFLWETYFSTADSIFNTVLAMIRTRIQIPAAPIREEAAAEIPHRSGKAAGTDPNMADNGDLDLGPVETEPPYASPRYLRNFTLTVTDFYRSWNCAPGPFHLFPHTPFDPVLPSEAKFLGSGTGFRPIGGGAGGSGKEFQAALGGNVPREQFTVVMLTYEREEVLMNSLERLNGLPYLNKVVVVWNSPKLPSEDLLWPDIGVPIMVVRTEKNSLNNRFLPWNEIETEAILSIDDDAHLRHDEIMFGFRVWREARDRIVGFPGRYHAWDIPHQSWLYNSNYSCELSMVLTGAAFFHKYYAYLYSYVMPQAIRDMVDEYINCEDIAMNFLVSHITRKPPIKVTSRWTFRCPGCPQALSHDDSHFHERHKCINFFVKVYGYMPLLYTQFRVDSVLFKTRLPHDKTKCFKFI